SEQUENCE LISTING (2 sequences)

SEQ ID NO: 1 - protein, 167 aa, Artificial sequence
  synthesized
MSEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTD

SEQ ID NO: 2 - protein, 1763 aa, Artificial sequence
  synthesized
SEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTDSGGSSGGSSGSETPGTSESATPESSGGSSGGSSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTDSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

(19) United States
(12) Patent Application Publication
(10) Pub. No.: US 2021/0395812 A1
(43) Pub. Date: Dec. 23, 2021

(54) METHOD FOR DETECTING OFF-TARGET EFFECT OF ADENINE BASE EDITOR SYSTEM BASED ON WHOLE-GENOME SEQUENCING AND USE THEREOF IN GENE EDITING

(71) Applicant: SUN YAT-SEN UNIVERSITY, Haizhu District, Guangzhou, Guangdong (CN)

(72) Inventors: Zhou SONGYANG, Haizhu District, Guangzhou, Guangdong (CN); Puping LIANG, Haizhu District, Guangzhou, Guangdong (CN); Junjiu HUANG, Haizhu District, Guangzhou, Guangdong (CN)

(21) Appl. No.: 17/279,124

(22) PCT Filed: Sep. 23, 2019
(86) PCT No.: PCT/CN2019/107243

§ 371 Date: Mar. 24, 2021

(30) Foreign Application Priority Data
Sep. 30, 2018 (CN) 201811160230.9

(51) Int. Cl. C12Q 1/6869

(52) U.S. Cl. CPC C12Q 1/6869

(57) ABSTRACT

The present invention provide a method for detecting the genome-wide off-target effects of adenine base editor (ABE) and the application in gene editing thereof. ABE comprises the TadA:TadA*:Cas9 fusion protein and gRNA which is able to catalyze the substitution of A to G with high efficiency at the target site, which can bring ABE a bright application prospect in gene editing and construction of disease model for human disease. Thus, the present invention provides the EndoV-seq method first time to detect the genome-wide off-target effects of ABE. The EndoV-seq method has a wide application prospect in gene editing, especially in gene editing for treatment field of human disease.

### CROSS-REFERENCE TO RELATED APPLICATIONS

The present application claims the benefit of priority to Chinese Patent Application No. 201811160230.9, filed on Sep. 30, 2018, entitled “METHOD FOR DETECTING THE GENOME-WIDE OFF-TARGET EFFECTS OF ADENINE BASE EDITOR AND APPLICATION THEREOF IN GENE EDITING”, the entire disclosures of which are hereby incorporated herein by reference.

### TECHNICAL FIELD

The technical field of the present invention belongs to molecular biology. More specifically, relates to a method for detecting the genome-wide off-target effects of adenine base editor (ABE) and application thereof in gene editing.

### BACKGROUND

The CRISPR/Cas9 system is a new artificial nuclease technology, which is a complex composed of gRNA (Guide RNA) and Cas9 protein (the Cas9-gRNA complexes). Under the help of 3′end PAM (Protospacer adjacent motif) sequence of the target site, the Cas9-gRNA complexes is combined with the target DNA through 20 bases at the 5′end of the gRNA, so that the endonuclease Cas9 is recruited to the target site, so the target DNA is cut, and the target gene is edited. Because the appearance of the CRISPR/Cas9 technology, the gene fixed-point mutation efficiency has been greatly improved, but the need of clinical gene therapy cannot be met at present. Recently, based on CRISPR/Cas9 technology, scientists have developed a new generation of gene editing system-adenine base editor (ABE). ABE is composed of TadA:TadA*:Cas9 fusion protein and gRNA (ABE-gRNA complexes). Under the guidance of gRNA, TadA:TadA*:Cas9 fusion protein can be combined with a target site on DNA, wherein the DNA strand complementary to the gRNA is nicked by Cas9 nuclease, and the A base of 4-9 positions on the non-complementary strand is catalyzed deamination to form an I base by tRNA-specific adenine deaminase (i.e., TadA). With the replication of DNA, I (Inosine) base is replaced by G (Guanine) base, thereby achieving base substitution of A to G. Compared with CRISPR/Cas9 nuclease, ABE shows higher efficiency. Since ABE can realize base substitution of A to G without inducing DNA double-strand break (DSB), the safety of ABE is higher than that of CRISPR/Cas9 nuclease.

About 48% of human pathogenic single base mutation can be repaired through base substitution of A to G, so that the treatment of genetic diseases is finally realized, and ABE has a wide application prospect in the field of human disease gene therapy. However, the method for detecting the genome-wide off-target effects of ABE is still not available at present, and the application of ABE is restricted severely.

### SUMMARY

Since there is no method for detecting the genome-wide off-target effects of ABE, the present invention aims at providing a method for detecting the genome-wide off-target effects of ABE and application of the method in gene editing.

According to one embodiment of the present invention, the off-target effects of ABE is detected by the techniques including gene synthesis, molecular cloning, protein expression and purification, in-vitro transcription, nucleic acid purification, whole-genome sequencing, PCR product depth sequencing, bioinformatics analysis and the like. Meanwhile, the detecting effectiveness and sensitivity of the method are verified by combining cell transfection and PCR product depth sequencing technology.

The aim of the present invention is realized by the following technical solutions:

A first aspect of the present invention provides a method for detecting the genome-wide off-target effects of adenine base editor, which comprising the following steps:

(1) TadA:TadA*:Cas9 fusion protein, one or more kinds of gRNA targeting to DNA to be detected, and genomic DNA comprising the DNA to be detected are blended and then subjected to reaction; wherein,

in the reaction system, strand of the DNA to be detected that complementary to the gRNA is nicked and the Adenine on the non-complementary strand is converted to Inosine by the ABE-gRNA complexes composed of the TadA:TadA*:Cas9 fusion protein and the gRNA;

(2) adding Endonuclease V into the system after reaction in the step (1), and cutting the DNA containing Inosine to cause DNA double-strand break;

(3) the off-target effects of adenine base editor is detected by using whole-genome sequencing and bioinformatics analysis.

According to one embodiment of the first aspect of the present invention, the present invention provides a method (named EndoV-seq) for detecting the genome-wide off-target effects of adenine base editor. The EndoV-seq first utilizes TadA:TadA*:Cas9 fusion protein purified in vitro and gRNA co-treat genomic DNA; the DNA strand complementary to the gRNA is nicked and the Adenine on the non-complementary strand is converted to Inosine by the complex of TadA:TadA*:Cas9 fusion protein and gRNA (the ABE-gRNA complexes); then the genomic DNA containing the I base is cut by endonuclease V (Endonuclease V, EnodV) to cause double-strand DNA break; and finally, the double-strand DNA fracture is detected by using the whole-genome sequencing and bioinformatics analysis, so that the off-target effects of ABE is explored. We name this method as EndoV-seq.

According to a specific embodiment of the first aspect of the present invention, the TadA:TadA*:Cas9 fusion protein comprises an effector protein domain of CRISPR/Cas9 system and an adenosine deaminase domain.

According to a specific embodiment of the first aspect of the present invention, the TadA:TadA*:Cas9 fusion protein comprises an effector protein domain of CRISPR/Cas9 system, a polypeptide linker and an adenosine deaminase domain.

A person skilled in the art can appreciate that the TadA:TadA*:Cas9 fusion protein of the present invention is formed by fusion of Cas9 effect protein and adenosine deaminase (TadA protein). A person skilled in the art can connect a Cas9 effector protein domain with one or more TadA proteins using one or more polypeptide linker according to needs, and obtain a fusion protein. In a specific embodiment of the present invention, the TadA protein is repeated once. It will be appreciated that the connecting order of the N-terminal and the C-terminal of the Cas9 effector protein and TadA protein is a conventional technique in the art, and the polypeptide linker comprises, but is not limited to, a conventional polypeptide linker fragment in the art, commonly, such as a GS linker.

A person skilled in the art can understand that any specific gRNA targeting to the genomic DNA can be designed according to specific needs, and the gRNA can be modified to improve the target specificity of the gRNA. in a specific embodiment of the present invention, the gRNA sequence designed by the present invention comprises sequence selected from the group consisting of (i) or (ii):

(i) HBG: GTGGGGAAGGGGCCCCCAAGAGG, wherein the underlined label is a PAM sequence.

(ii) VEGFA3: GGTGAGTGAGTGTGTGCGTGTGG, wherein the underlined label is a PAM sequence.

A person skilled in the art can understand that as for TadA:TadA*:Cas9 fusion protein, TadA is an abbreviation of adenosine deaminase, TadA* is an abbreviation of TadA mutant, and Cas9 is a Cas9 effector protein of a CRISPR/CAS system.

Furthermore, the Cas9 effector protein in the effector protein domain of the CRISPR/Cas system comprises, but is not limited to, a CAS protein with no cleavage activity or only single strand cleavage activity, such as Streptococcus pyogenes Cas9 (SpCas9), Staphylococcus aureus Cas9 (SaCas9), Lachnospiraceae Cpf1 (LbCpf1), Acidaminococcus Cpf1 (AsCpf1), Streptococcus thermophilus Cas9 (StCas9), and Neisseria meningitidis Cas9 (NmCas9) and Francisella Cpf1 (FnCpf1), etc.

Furthermore, the amino acid sequence of the adenosine deaminase TadA protein of the TadA:TadA*:Cas9 fusion protein comprises SEQ ID NO.1.

In a specific embodiment of the first aspect of the present invention, the amino acid sequence of the TadA:TadA*:Cas9 fusion protein comprises SEQ ID NO.2 or a sequence consistent with at least 80%, 85%, 90%, 92%, 95%, 96%, 97%, 98%, 99% or 99.5% of the amino acid sequence shown in SEQ ID NO.2.

According to one embodiment of the first aspect of the present invention, the preparation of the TadA:TadA*:Cas9 fusion protein comprise: TadA:TadA*:Cas9 fusion protein is expressed by a prokaryotic expression vector (pET42b-ABE7.10, SEQ ID NO.3) in E. coli and purified.

Furthermore, the prokaryotic expression vector is constructed by molecular clone.

More specifically, the preparation process comprises:

step (1), comprises: transforming the pET42b-ABE7.10 into BL21 Star™ (DE3) E. coli (Thermo Fisher) competent cells.

More specifically, step (2), inducing the expression of the TadA:TadA*:Cas9 fusion protein, comprises: a single clone is picked up and cultured overnight at 37° C., then inoculated according to a ratio of 1:200 in LB culture medium containing 50 μg/ml kanamycin, and cultured at 37° C. until OD600 of 0.7-0.8. Then, the culture solution is stand for 1 hour at 4° C. in a refrigerator before addition of IPTG with a final concentration of 0.5 mM and induction at 18° C. for 14-16 h.

More specifically, step (3), purifying and preserving the TadA:TadA*:Cas9 fusion protein, comprises: the induced bacteria solution is collected at 4000 rpm, 10 min, and lysis in 10 ml lysis solution (100 mM Tris-HCl, pH 8.0, 1 M NaCl, 20% glycerol, 5 mM TCEP (Sigma-Aldrich), 0.4 mM PMSF (Sigma-Aldrich), protease inhibitor (Roche) and 20 mM Imidazole (Sigma-Aldrich)), followed by a preliminary sonication (5 min total, 2 s on, 5 s off) to crush cells, then the supernatant is collected (at 15000 rpm, 4° C.) and followed by a second sonication (5 min total, 2 s on, 5 s off), and again the supernatant is collected (at 15000 rpm, 4° C.). The supernatant is then incubated with Ni-NTA agarose resin (GE Healthcare) for 1.5 hour at 4° C. and then the mixed solution is poured into a chromatographic column and washed in 40 ml wash buffer (100 mM Tris-HCl, pH8.0, 0.5 M NaCl, 20% glycerol, 5 mM TCEP, and 20 mM imidazole) before the protein is eluted from the Ni column (100 mM Tris-HCl, pH8.0, 0.5M NaCl, 20% glycerol, 5 mM TCEP, and 270 mM imidazole). All eluted proteins are further purified on a 5 mL Hi-Trap HP SP cation exchange column (GE Healthcare), concentrated with the 30 kDa Centrifugal Filter Unit (Millipore), sterile filtered (0.22 μm PVDF membrane)(Millipore), and quantified using the BCA assay (Pierce Biotechnology). The purified proteins are stored at 4° C. and snap-frozen in liquid nitrogen for storage at −80° C. as for long-term storage.

More specifically, the method for preparing gRNA comprises the following steps: (1) chemically synthesizing gRNA; (2) synthesizing gRNA by in-vitro transcription.

In a specific embodiment of the first aspect of the present invention, the reaction system is a solution reaction system, and the solution reaction system further comprises one or more buffer solution components required for converting adenine on the non-complementary strand into Inosine by the TadA:TadA*:Cas9 fusion protein.

In a specific embodiment of the first aspect of the present invention, the step (3) comprises:

performing whole-genome sequencing on the production subjected to enzyme digestion in the step (2) to obtain a whole-genome sequencing result;

performing bioinformatics analysis on the whole-genome sequencing result to obtain off-target data of adenine base editor.

Furthermore, the step (3) further comprises: predicting the off-target effects of adenine base editor in cells (including human cells, animal cells, plant cells, etc.) or in body (including humans, animals, plants, etc.) according to the off-target data.

According to the present invention, the TadA:TadA*:Cas9 fusion protein purified in-vitro and gRNA is used in the present invention, and the ABE-gRNA complexes is used to treat the genomic DNA. Then, the treated genomic DNA is purified using a nucleic acid purification kit and digested with endonuclease V, and purified again. Then the purified genomic DNA is performed with whole-genome sequencing to detect the genome-wide off-target effects of ABE. The method and the result of EndoV-seq do not depart from the scope of the present invention. At the same time, the gRNA with high efficiency and specificity can also be obtained according to the result of EndoV-seq. It is also within the scope of the present invention to provide a method for selecting preferred gRNA for EndoV-seq, for further experiments to verify the off-target effects of adenine base editor in vivo (such as Example 3) or for following gene editing application.

In a specific embodiment of the first aspect of the present invention, choosing gRNAs with low off-target effects according results of whole-genome sequencing and bioinformatics analysis for gene editing or for verifying the off-target effects of adenine base editor in vivo.

Further, the sequence of the selected gRNA comprises sequence selected from the group consisting of (i) or (ii):

(i) HBG: GTGGGGAAGGGGCCCCCAAGAGG, wherein the underlined label is a PAM sequence.

(ii) VEGFA3: GGTGAGTGAGTGTGTGCGTGTGG, wherein the underlined label is a PAM sequence.

In a specific embodiment of the first aspect of the present invention, the detecting efficiency of the method for detecting the off-target effects of adenine base editor in vivo can be at least low to 0.13%.

Furthermore, the detecting efficiency of the method for detecting the off-target effects of adenine base editor in vivo at HBG-OT9 site can be at least low to 0.13%.

According to one embodiment of the first aspect of the present invention, one specific application including searching for gRNAs with high efficiency and specificity targeting to the DNA to be detected.

In a specific embodiment of the first aspect of the present invention, the step (3) comprises:

performing whole-genome sequencing on the production subjected to enzyme digestion in the step (2) to obtain a whole-genome sequencing result;

performing bioinformatics analysis on the whole-genome sequencing result to obtain off-target data of adenine base editor;

choosing gRNAs with low off-target effects according results of whole-genome sequencing and bioinformatics analysis;

expressing the TadA:TadA*:Cas9 protein and gRNA in cells, the extracting the genomic DNA which is then depth sequenced to predict the off-target effects of adenine base editor in cells (including human cells, animal cells, plant cells, etc.) or in body (including humans, animals, plants, etc.) according to the off-target data.

According to the present invention, the application of the method for detecting the genome-wide off-target effects of adenine base editor in cells is of course within the scope of the present invention.

According to the present invention, EndoV-seq is also able to detecting the efficiency and off-target effects of other enzymes or chemical agents which are capable of converting the base A to base I. Such enzymes include but not limited to TadA adenosine deaminase provided by the first aspect of the present invention.

According to the present invention, Endonuclease V used in EndoV-seq can also be replaced by other endonuclease that has the same digesting site as Endonuclease V, which is of course within the scope of the present invention.

A second aspect of the present invention provides a kit for detecting the genome-wide off-target effects of adenine base editor. which comprises the gRNA targeting DNA to be detected, TadA:TadA*:Cas9 fusion protein and the EndoV nuclease provided by the first aspect.

A third aspect of the present invention provides an application of the method for in gene editing.

According to the present invention, the application of EndoV-seq in gene editing is also within the scope of the present invention.

According to the present invention, ABE will be used as a tool to promote clinical application, such as accurate gene editing treatment, construction of a precise disease model, or plant or crop cultivation by accurate gene editing and the like.

Advantageous effects are provided by technical solutions of the present disclosure, that is:

The present invention provides a method for detecting the genome-wide off-target effects of adenine base editor, which is able to detect the off-target effects of ABE, and promote widely application of ABE, including in gene treatment for diseases, construction of disease model, or plant or crop cultivation by gene editing and the like.

### BRIEF DESCRIPTION OF THE DRAWINGS

FIG. 1 is a protein gel electrophoresis diagram of Cas9 protein, BE3 protein, and TadA:TadA*:Cas9 protein; the first lane is protein molecule Marker, the second lane is Cas9 protein, the third lane is BE3 protein, and the fourth lane is TadA:TadA*:Cas9 protein.

FIG. 2 is a gRNA agarose gel electrophoresis result, both lanes are gRNA.

FIG. 3 presents the co-treatment of the ABE-gRNA complexes and EndoV being able to cut the target DNA molecule.

FIG. 4 presents the capability of EnodV-seq to detect the DNA double-strand break at the target site.

FIG. 5 presents the EndoV-seq used to detect the genome-wide off-target effects of ABE mediated by the HBG gRNA and VEGFA3 gRNA. A graph shown by Circos Plot illustrating the target sites and off-target sites detected by EndoV-seq, the target site is indicated by the arrow. B graph illustrates the molecular pattern of the off-target sites shown by Weblog. The target sequence of gRNA is labeled below.

FIG. 6 presents the off-target sites of HBG verified by depth sequencing using the PCR product, with 6 sites in 18 off-target sites being verified and marked with *.

FIG. 7 presents the off-target sites of VEGFA3 verified by depth sequencing using the PCR product, with 3 sites in 22 off-target sites being verified and marked with *.

FIG. 8 is a flowchart of a method for detecting the genome-wide off-target effects of adenine base editor.

### DETAILED DESCRIPTION OF ILLUSTRATED EMBODIMENTS

The present disclosure will be described further below with reference to drawings and specific embodiments, but the embodiments are not intended to limit any form of the present invention.

Unless specifically defined, reagents, methods, and devices employed in the present invention are conventional reagents, methods, and devices in the art. Unless specifically stated, the reagents and materials used in the following examples are commercially available. Experimental methods for specific conditions are not noted, typically carried out according to conventional conditions, or at the manufacturer's suggested conditions.

In a specific embodiment of the present invention, the present invention provides a platform for detecting the genome-wide off-target effects of adenine base editor, and method, kit, application thereof.

According to the kit provided by the present invention, the implementation of the method for detecting the genome-wide off-target effects of adenine base editor comprises but not limit to the following one or more steps:

### Example 1

Expression and Purification of the TadA:TadA*:Cas9 Fusion Protein, Preparation of gRNA

1, The Expression and purification of the TadA:TadA*:Cas9 fusion protein

Preparation of a recombinant expression plasmid containing a gene encoding TadA:TadA*:Cas9 fusion protein, in this embodiment, the prokaryotic expression vector containing a gene encoding TadA:TadA*:Cas9 fusion protein is pET42b-ABE7.10 (SEQ ID NO.3);

Step (1), A BL21 Star™ (DE3) E. coli (Thermo Fisher) competent cell was transformed with the pET42b-ABE7.10.

Step (2), Inducing the expression of the TadA:TadA*:Cas9 fusion protein: a single clone was picked up and cultured at 37° C. overnight, then inoculated according to a ratio of 1:200 in LB medium containing 50 μg/ml kanamycin, and after cultured overnight until OD600 of 0.7-0.8, the culture solution was stand for 1 hour at 4° C. in a refrigerator before addition of IPTG with a final concentration of 0.5 mM and induction at 18° C. for 14-16 h.

Step (3), Purification and Preservation of the TadA:TadA*:Cas9 fusion protein, the BL21 cells was collected at 4° C. and the protein was purified: the BL21 cells induced were collected (4000 rpm, 10 min) and lysed in 10 ml lysis buffer (100 mM Tris-HCl, pH8.0, 1M NaCl, 20% glycerol, 5 mM tris (2-carboxyethyl) phosphine (TCEP; Sigma-Aldrich), 0.4 mM PMSF (Sigma-Aldrich), protease inhibitors (Roche), and 20 mM imidazole (Sigma-Aldrich)) followed by a preliminary sonication (5 min total, 2 s on, 5 s off) to crush cells, then the supernatant was collected (at 15000 rpm, 4° C.) and followed by a second sonication (5 min total, 2 s on, 5 s off), and again the supernatant was collected (at 15000 rpm, 4° C.). The supernatant was then incubated with Ni-NTA agarose resin (GE Healthcare) for 1.5 hour at 4° C. and then the mixed solution was poured into a chromatographic column and washed in 40 ml wash buffer (100 mMTris-HCl, pH8.0, 0.5M NaCl, 20% glycerol, 5 mM TCEP, and 20 mM imidazole) before the protein was eluted from the Ni column (100 mM Tris-HCl, pH8.0, 0.5M NaCl, 20% glycerol, 5 mM TCEP, and 270 mM imidazole).All eluted proteins were further purified on a 5 mL Hi-Trap HP SP cation exchange column (GE Healthcare), concentrated with the 30 kDa Centrifugal Filter Unit (Millipore), sterile filtered (0.22 μm PVDF membrane)(Millipore), and quantified using the BCA assay (Pierce Biotechnology). The purified proteins were stored at 4° C. and snap-frozen in liquid nitrogen for storage at −80° C. as for long-term storage.

The detecting results of protein expression are shown in FIG. 1, FIG. 1 is a diagram showing the protein gel electrophoresis result of the Cas9 protein, the BE3 protein and the TadA:TadA*:Cas9 fusion protein.

2, the Preparation of gRNA

The gRNA in the embodiment of the present invention was prepared directly by chemical synthesis method or in vitro transcription method, wherein the in vitro transcription method to prepare the gRNA including the following steps: \textcircled{1} the gRNA transcription template DNA containing T7 promoter was prepared by PCR. Alternatively, the gRNA coding sequence was cloned into a transcription vector containing T7 promoter and the vector was then linearized to obtain a gRNA transcription template DNA containing T7 promoter; \textcircled{2} the gRNA was transcript in vitro.

The method of transcription of the gRNA in vitro including: The gRNA transcription template DNA containing the T7 promoter was used as a template, and the gRNA is produced using MegaShortScript T7 Kit (Life Technologies). The gRNA was purified by RNA purification kit (Qiagen), and eluted with water without nuclease.

Specifically, the operation procedure of the method of transcription of the gRNA in vitro was as follows:

1) Using the gRNA transcription template DNA as a template and MEGAshortscript T7 kit (Life Technologies), a reaction system was formulated according to the system shown in Table 1 below.

TABLE 1
the reaction system

| Ingredients | Usage |
|---|---|
| T7 10 × Reaction Solution | 2 μl |
| T7 ATP Solution | 2 μl |
| T7 CTP Solution | 2 μl |
| T7 GTP Solution | 2 μl |
| T7 UTP Solution | 2 μl |
| DNA Template | 1 μg |
| T7 RNA Transcriptase | 2 μl |
| ddH2O | add water to 20 μl |

Reaction for 2 h at 37° C., then 1 μl TURBO DNase was added to the reaction system, and then reacting for 15 min at 37° C.

2) Purification of the gRNA by the RNEasykit of Qiagen, including the following steps:

a. ddH2O was added so that the volume of the starting RNA was 100 μl, and mixed evenly.

b. 350 μl of Binding Solution Concentration was added into the RNA samples and mixed evenly.

c. 250 μl ethanol (100%) was added and mixed evenly.

d. The sample was transferred into a column, centrifuged at 12,000 g for 15 s.

e. The sample was washed twice with 500 μl Wash Solution, then centrifuged at 12,000 g for 15 s.

f. The RNA was eluted from the column by 50 μl ddH2O.

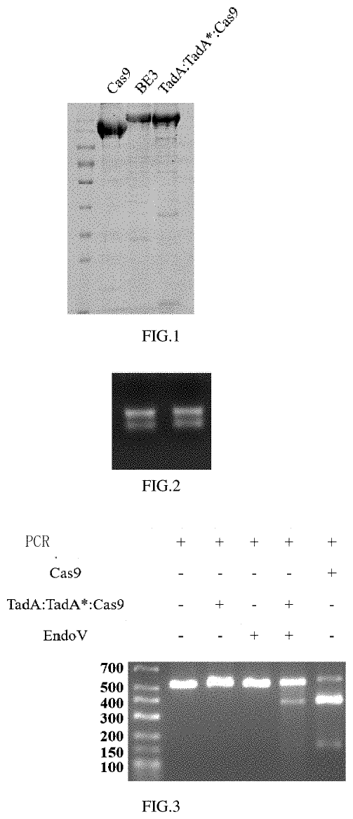

3) The results are shown in FIG. 2, FIG. 2 is a diagram showing the agarose gel electrophoresis result of the HBG gRNA and VEGFA3 gRNA.

Using EndoV-Seq to Detect the Single-Base Editing on Target Sites by ABE

In order to verify whether the EndoV-seq can be used to detect the genome-wide off-target effects of ABE, the HEK293-2 gRNA, the sequence of which is GAACCAAAGCATATGTGCGGG with underlined labeled PAM sequences, which has been verified multiple times to efficiently target the target site was used. First, the PCR products containing HEK293-2 sites were amplified by PCR and then purified, the specific purification method was as follows.

Experiments were conducted according to the operation manuals of the AxyPrep PCR cleanup kit

a, in a PCR reaction solution, three volumes of Buffer PCR-A were added and mixed evenly, then transferred to a DNA preparation tube, the DNA preparation tube was placed in a 2 ml centrifuge tube, then centrifuged for 1 min at 12,000 g, and the filtrate was discarded.

b. The preparation tube was put back into a 2 ml centrifuge tube, 700 μl Buffer W2 was added, then centrifuged for 1 min at 12,000 g, and the filtrate was discarded.

c. The preparation tube was put back into a 2 ml centrifuge tube, 400 μl Buffer W2 was added, then centrifuged for 1 min at 12,000 g, and the filtrate was discarded.

d. The preparation tube was then centrifuged for 3 min, so that the ethanol in Buffer W2 was sufficiently discarded.

e. The preparation tube was placed in a new 1.5 ml centrifuge tube, 25-30 μl nuclease-free water was added in the center of the preparation tube, and stand for 1 min. Then the centrifuge tube was centrifuged for 1 min at 12,000 g. (Pre-heating the nuclease-free water first at 65° C.).

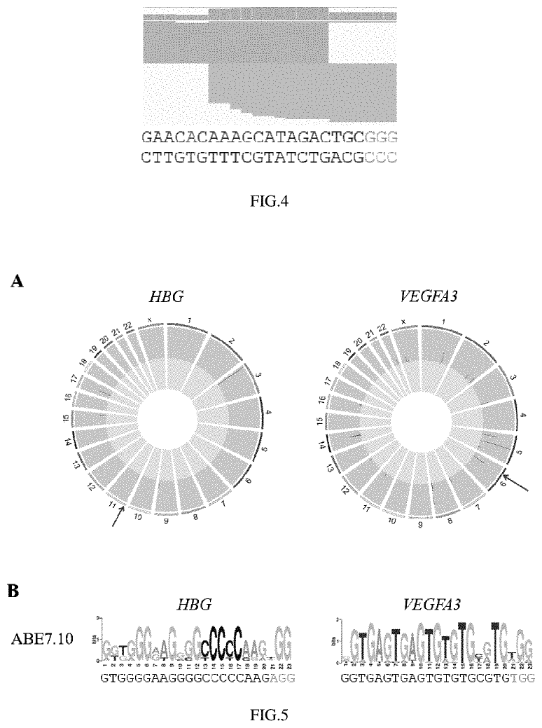

After the purified PCR products were prepared, the PCR products were added to a 20 μl reaction system. The reaction system contained 2 μl of 10×NEBuffer 3, 400 nM TadA:TadA*:Cas fusion protein, 900 nM gRNA and 200 ng PCR product. RNase A and Protease K were added in turn to remove the gRNA and protein after reaction at 37° C. for 3 h. The reaction system was then purified again according to the above steps. 100 ng purified product and 1 unit Endo V (Thermofisher) were mixed and reacted at 65° C. for 30 min. The products were resolved on a 3% agarose gel. The results are shown in FIG. 3. The PCR products containing HEK293-2 target sites, which had been treated by TadA:TadA*:Cas9 and gRNA, could be cut off by EndoV. Wherein the Cas9 protein was used as a positive control. FIG. 3 illustrates that the EndoV can be used to detect the deamination of ABE.

To further detect whether the EndoV-seq can be used to detect the deamination of ABE, the genomic DNA of human HEK293T cells were further treated with TadA:TadA*:Cas9 fusion protein and HEK293-2 gRNA. First, the genomic DNA was extracted from HEK293T cells using genomic DNA extraction kit (DNeasy Blood & Tissue Kit, Qiagen), the extracting steps were performed specifically according to the description. The genomic DNA of human HEK293T cells was then treated with TadA:TadA*:Cas9 fusion protein and HEK293-2 gRNA. 50 μl 10×NEBuffer 3, 400 nM ABE7.10, 900 nM gRNA and 10 μg genomic DNA were added in a 500 μL reaction system. After reaction for 8 hours at 37° C., RNase A and Protease K were added to the reaction system to remove gRNA and protein. The genomic DNA was then extracted with phenol/chloroform/isoamyl alcohol, the operational steps were as follows.

a. 1 volume of phenol/chloroform/isoamyl alcohol was added to the above reaction and mixed vigorously, stand at room temperature for 10 minutes, and centrifuged at 12000 rpm for 10 minutes after layered;

b. the upper water phase layer was sucked and its volume was recorded;

c. 1/10 volume of 3M NaAc and 3-fold volume cold anhydrous ethanol (stored in −20° C. refrigerator) were added, mixed vigorously. Then incubated on ice for 15 minutes;

d. then centrifuged (12000 rpm, 15 min, 4° C.), afterwards the ethanol was removed as much as possible with a pipette;

e. 0.5 ml 70% ethanol was added to wash DNA precipitate once, then centrifuged at 12000 rpm for 2 minutes, the ethanol was drawed and discarded as much as possible;

f. 30 μl water was added to dissolve the DNA and then the concentration is detected with Nanodrop.

Then, 4 μg genomic DNA and 8 units EndoV nuclease (ThermoFisher) were mixed in a 100 μl reaction system and reacted at 65° C. for 3 hours, and the genomic DNA was extracted with phenol chloroform. Finally, whole-genome sequencing was carried out using 1 μg genomic DNA. The sequenced reads were then aligned to the human reference genome sequences with BWA software. We found that the EndoV-seq surely could be able to detect ABE-mediated modification of the target site, as shown in FIG. 4.

### Example 2

### EndoV-Seq to Detect the Genome-Wide Off-Target Effects of ABE

To further explore whether the EndoV-seq can be used to detect the genome-wide off-target effects of ABE. We further utilized the HBG and VEGFA3 gRNAs that were prepared in Example 1, and then incubated with the TadA:TadA*:Cas9 fusion protein, respectively. The HEK293-2 genomic DNA was then treated with the protein-RNA complex.

50 μl 10×NEBuffer 3, 400 nM ABE7.10, 900 nM gRNA and 10 μg genomic DNA were added to a 500 μl reaction system. After reacting for 8 hours at 37° C., RNase A and Proteinase K were added to the reaction system to remove gRNA and proteins. Genomic DNA was then extracted with phenol/chloroform/isoamyl alcohol, and the operational steps were as follows.

a. 1 volume of phenol/chloroform/isoamyl alcohol was added to the above reaction and mixed vigorously, stand at room temperature for 10 minutes, and centrifuged at 12000 rpm for 10 minutes after layered;

b. the upper water phase layer was sucked and its volume was recorded;

c. 1/10 volume of 3M NaAc and 3-fold volume cold anhydrous ethanol (stored in −20° C. refrigerator) were added, mixed vigorously. Then incubated on ice for 15 minutes;

d. then centrifuged (12000 rpm, 15 min, 4° C.), afterwards the ethanol was removed as much as possible with a pipette;

e. 0.5 ml 70% ethanol was added to wash DNA precipitate once, then centrifuged at 12000 rpm for 2 minutes, the ethanol was drawed and discarded as much as possible;

f. 30 μl water was added to dissolve the DNA and then the concentration is detected with Nanodrop.

Then, 4 μg genomic DNA and 8 units EndoV nuclease (ThermoFisher) were mixed in a 100 μl reaction system and reacted at 65° C. for 3 hours, and the genomic DNA was extracted with phenol chloroform. Finally, whole-genome sequencing was carried out using 1 μg genomic DNA. The sequenced reads were then aligned to the human reference genome sequences with BWA software. The genomic DNA cleavage was assessed using the Digenome2.0 tool(http://www.rgenome.net/digenome-js/standalone) and the cleavage score for each target position were calculated. We defined a site with score above 0.1 as the positive off-target site, according to the study which using Digenome-seq to detect the off-target effects of the cytosine single-base editing system. We found that the EndoV-seq could be able to identify the target site and the off-target sites, the results were shown in FIG. 5, the off-target sites identified by EndoV-seq were showed in table 2 and table 3.

TABLE 2
off-target sites of the HBG gRNA guided ABE identified by EndoV-seq

| | | | | deep sequencing primers | |
|---|---|---|---|---|---|
| site | position | sequence | score | FP | RP |
| HBG1-TA | chr11:5271278 | GTGGGGAAGGGG | 2.0 | TCCTGGTATCCTC | GTGGAGTTTAGCC |
| | | CCCCCAAGAGG | | TATGATGGGA | AGGGACC |
| HBG2-TA | chr11:5276202 | GTGGGGAAGGGG | 1.4 | TGGTGGGAGAAG | GTGGAGTTTAGCC |
| | | CCCCCAAGAGG | | AAAACTAGC | AGGGACC |
| HBG-OT1 | chr3:13705838 | GgtGGGAtGGGGtC | 11.4 | ATGGCTGCAAATC | AAATGCTTCTCGG |
| | | CCCAAGTGG | | CAAGGGT | GCTCTCC |
| HBG-OT2 | chr17:35304222 | GgtaGGgAGaGGCC | 4.8 | GAGGTTGAAACT | GGAATTAAGATGC |
| | | CCCAgaGGG | | CCTCGCCA | AACTGAGAGTAG |
| HBG-OT3 | chr9:138419302 | GgtGGGgAGcGGC | 2.8 | GGCTGTCCCTGGT | CGAGCACTGAGGC |
| | | CCCCcAGTGG | | TGTCTGG | CTGGTTA |
| HBG-OT4 | chr15:84049561 | agtGGGgAGGcGCC | 2.5 | GTGCCTTGGCTTG | TTCTGGGAGGAGA |
| | | CtCAAGTGG | | CTATTTGG | GTTGGGGT |
| HBG-OT5 | chr10:73282210 | GTGGGG-AGtGGC | 2.2 | TATTATATCCATT | CACACAGATGTAA |
| | | CCCCAAGAGG | | CCAGTGGGTTTC | GTGGTAAGAGCA |
| HBG-OT6 | chr9:138419300 | GTGGGG-AGcGGC | 2.2 | GCAGAAAGTGTA | CTTCCTGAGCAGC |
| | | CCCCcAGTGG | | GCGGGTGAC | TGATTGGT |
| HBG-OT7 | chr19:33976063 | GTGaGGGgAGGGa | 2.1 | ACTGGCTTCTTTC | GCTGGGATTACAG |
| | | CCCtCAAGAGG | | CGGGC | GTGTGAGTC |
| HBG-OT8 | chr7:88444871 | GTGGGGAAaGGtG | 2.0 | GTCCCTTGTATGT | TGTCAGAGCCTGA |
| | | CCCaCAAGGGG | | AACCAATCTCAC | GAAGGTGAA |
| HBG-OT9 | chr3:4746491 | aTGaGGAAGcGaC | 1.7 | GTTGATGTCTGTG | GTACACCCGTCAC |
| | | CCCCAAGAGG | | GAACGGT | AGCTAGA |
| HBG-OT10 | chr5:117819228 | GTGGGGtAaGaaCC | 1.4 | GATGGTGGCCCTC | GGATGCCCAGACA |
| | | CCCAAaTGG | | TTCTCACA | AAAGTATGC |
| HBG-OT11 | chr9:21122154 | GgtGaGAAGGaGC | 1.0 | TTGTTATAGAGGA | TTTGCTTAACATA |
| | | CCaCAAGTGG | | ACCCCAGCC | CAGAGTTCCTG |
| HBG-OT12 | chr3:34824512 | GgtaGGAAGGGGC | 0.7 | TTCCCTTACTGAT | AAACACTCTGTGA |
| | | tCCCAAGAGG | | CCGTGTCC | GTCACTTTTGA |
| HBG-OT13 | chrX:12298826 | GTGGGaAAGGacC | 0.7 | CCCATGAAGTCCC | CAGGTGGCTAGGC |
| | | CCCaAtGAGG | | CACTGTC | TGAAACA |
| HBG-OT14 | chr5:134498847 | GTGGaaAAGGaGa | 0.5 | GCGACACTTTTGC | CTCCACCCTATGA |
| | | CCCCAAGAGG | | TCTACGTG | CACCACCT |
| HBG-OT15 | chr17:7365166 | GTGtG-AAGGGGC | 0.4 | TCTCACACAAAGC | CTTTCACCAGATG |
| | | tCCCAAGGGG | | AGCCTGA | CCACCCT |
| HBG-OT16 | chr10:79889820 | GTGaGGAAGatGtC | 0.4 | AGTGTACTTTGCA | TCCTGAAGTCCAG |
| | | CCCAAGTGG | | AGCAAAAGGG | TTCCCTTCC |
| HBG-OT17 | chr7:2663814 | GTGaGGAcAGGGt | 0.4 | AACCTAGAGGGT | TGGAAAAGGGGTG |
| | | CCCCCAAGGGG | | GCAGGACA | TCCAAGG |
| HBG-OT18 | chr20:24950449 | aTGGGGAA-GGGC | 0.3 | GAAGAAGGTGAC | GAGGGGTATTCCT |
| | | CCCCAGGGGC | | TCCGCCTC | GTCCAGC |

TABLE 3
off-target sites of the VEGFA3 gRNA guided ABE identified by EndoV-seq

| | | | | deep sequencing primers | |
|---|---|---|---|---|---|
| site | position | sequence | score | FP | RP |
| VEGFA3-TA | chr9:110103695 | GGTGAGTGAGTG | 18.1 | GTGCAGACGGCA | CTATTGGAATCCT |
| | | TGTGCGTGTGG | | GTCACTAGG | GGAGTGACCC |
| VEGFA3-OT1 | chr5:89440969 | aGaGAGTGAGTG | 41.5 | GTGGGACCTGGTG | ACAATCATGGAA |
| | | TGTGCaTGAGG | | GGAGT | GAATGCAAAG |
| VEGFA3-OT2 | chr8:143890817 | GGTGtaTGAGTG | 28.7 | GTGTGACTAAGTG | GTGACGATTTATG |
| | | TGTGtGTGAGG | | TGAGAGTATGTG | CTAATGTGT |
| VEGFA3-OT3 | chr5:29367379 | tGTGAGTGAGTG | 20.3 | TTTTGTTTCTAAA | TGTTCCATTGTCT |
| | | TGTGtaTGGGG | | AATTAAACTAAG | GAAATGTAT |
| VEGFA3-OT4 | chr14:65569159 | aGTGAGTGAGTG | 15.7 | GCTCATTTCCTAC | CTGCAGTGAGGA |
| | | TGTGtGTGGGG | | GGCCCAG | GGTGGTTC |
| VEGFA3-OT5 | chr1:181557193 | GGaGAGTGAGTG | 14.9 | GTGGGTTTTGAAA | AATTGAGCTGAA |
| | | TGTGCaTGTGC | | CCAAATGTTCT | ACAGGACCAG |
| VEGFA3-OT6 | chr14:62078773 | tGTGAGTaAGTG | 11.2 | GCCACAGGCACT | GATGAAGCTGCCT |
| | | TGTGtGTGTGG | | AACTTCTTCA | TTCCTAAGC |
| VEGFA3-OT7 | chr3:193993884 | aGTGAaTGAGTG | 10.3 | CCCTTTGTGACCC | TAAGGCACGAGT |
| | | TGTGtGTGTGG | | AAAAGATTCC | CAGGATGGG |
| VEGFA3-OT8 | chr2:230506241 | GGTGAGcaAGTG | 9.2 | CAGATGCAAATG | AATGGATCAGAG |
| | | TGTGtGTGTGG | | CAAAAGAACAATA | GACCAACATTGTA |
| VEGFA3-OT9 | chr8:141037917 | aGTGAGTGAGTG | 7.7 | ATCATGGCAGAA | CACGTGTCGAGG |
| | | TGTGtGTGAAG | | GGGGAAGA | GAGGGAC |
| VEGFA3-OT10 | chr22:37662824 | GcTGAGTGAGTG | 7.2 | PCR failed | |
| | | TaTGCGTGTGG | | | |
| VEGFA3-OT11 | chr7:152671378 | aGTGAGTGAGTG | 7.2 | TGTATTTACTCCA | CCATGAAGTATGT |
| | | aGTGaGTGAGG | | TTCACCATCA | TCCATCTGA |
| VEGFA3-OT12 | chr2:177463426 | GGTGAGTGtGTG | 7.0 | GCGCTTTCCCTTT | CTCAGCAATGCTT |
| | | TGTGCaTGTGG | | GCTAGAATC | ATATTACTGGC |
| VEGFA3-OT13 | chrX:41726218 | GGTGAGTGAGTG | 7.0 | GCATACTAGACTA | TTCCCAAACAGTG |
| | | aGTGaGTGAGG | | GGGGTTCTGC | TGCCATGAT |
| VEGFA3-OT14 | chr11:79178512 | aGTGAGTGAGTG | 4.8 | TCCTTAATGTTTT | GAACCTCTAGTAG |
| | | aGTGaGTGGGG | | TGCATTGGAGG | GAGTCGCT |
| VEGFA3-OT15 | chr16:80016320 | tGTGAGTGAGTG | 4.0 | GCATTGGGTATTT | TGAGCCTTTGAAG |
| | | TGTGCGTGTGA | | GTGTGTATAA | TGTGTCTCT |
| VEGFA3-OT16 | chr12:114752926 | tGTGAGTGAGTG | 3.8 | CCTCTCAGACATG | CACACTCAAACAT |
| | | TGTGCaTGTGA | | GAAAGGG | GCTCACACA |
| VEGFA3-OT17 | chr5:150224710 | GGTGAGTGAGaG | 3.2 | ATGTTAGGGTGTG | ACAGCCACTCATA |
| | | TGTGtGTGTGG | | TGAACGTG | CCTGGTG |
| VEGFA3-OT18 | chrX:56327306 | tGTGAGTGtGTG | 3.0 | ATGAACACCCAC | TGACCTCTATTCC |
| | | TGTGCaTGTGG | | ATACCCTT | ACTCACTTT |
| VEGFA3-OT19 | chr6:157078327 | GaTGAGTGAGTG | 2.2 | AGTGTCCAGTGTT | TTAAAATGATTCA |
| | | aGTGaGTGGGG | | GATAAAGTCTA | CCTGTATAAGG |
| VEGFA3-OT20 | chr14:98442523 | GGTGAGTGtGTG | 2.0 | PCR Failed | |
| | | TGTGaGTGTGG | | | |
| VEGFA3-OT21 | chrX:42430834 | aGTGAGTGAGTG | 1.8 | ACATTGCTACACC | ACTGACAAGGTC |
| | | TGaGCGTGAAG | | TTTGGATTCT | ATTTGATTGGAC |
| VEGFA3-OT22 | chr5:115434669 | tGTGgGTGAGTG | 1.8 | CAATGTGATGATT | TCTAATGTATGGC |
| | | TGTGCGTGAGG | | TGATAGCTG | ATGGTGACT |
| VEGFA3-OT23 | chr10:109378067 | GGTGAGTGAGTG | 1.4 | AAAGTCTGTGGTA | ATATAGTATAAG |
| | | aGTGaGTGAGG | | GTGTATAGTAAT | AGATAAAAAATGG |
| VEGFA3-OT24 | chr6:24224733 | GGTGAGcGtGTG | 1.1 | GGGGTACAATGG | TGCCACCCCAGTT |
| | | TGTGCaTGTGG | | TGCACAGA | TTGAGTT |
| VEGFA3-OT25 | chr16:12264602 | aGTGAGTGAGTG | 1.0 | PCR failed | |
| | | TGTGtGTGTGA | | | |
| VEGFA3-OT26 | chrX:149380335 | aaTGAaTGAGTG | 0.5 | PCR failed | |
| | | aGTGtGTGAGT | | | |
| VEGFA3-OT27 | chr11:7625795 | GGTGAGTagGTG | 0.4 | PCR failed | |
| | | TGTGtGTGGGG | | | |
| VEGFA3-OT28 | chr10:107867368 | aGaGAGTGAGTG | 0.3 | PCR failed | |
| | | TGTGtGTtGGG | | | |
| VEGFA3-OT29 | chr12:5100948 | tGTGAaTGAGTG | 0.3 | PCR failed | |
| | | TGTGCaTGTGA | | | |
| VEGFA3-OT30 | chr1:47690894 | tGTGAGaGAGaG | 0.2 | PCR failed | |
| | | TGTGCGTGTGG | | | |
| VEGFA3-OT31 | chr5:150224714 | GGTGAGTGAGaG | 0.2 | PCR failed | |
| | | TGTGtGTGTGG | | | |
| VEGFA3-OT32 | chr1:4770551 | aagtgtgtgagt | 0.1 | PCR failed | |
| | | gtgtgcgtGTA | | | |

### Example 3

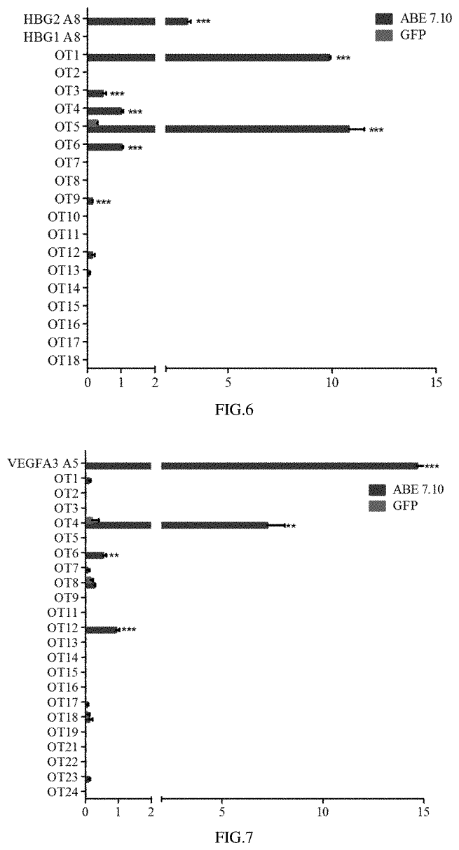

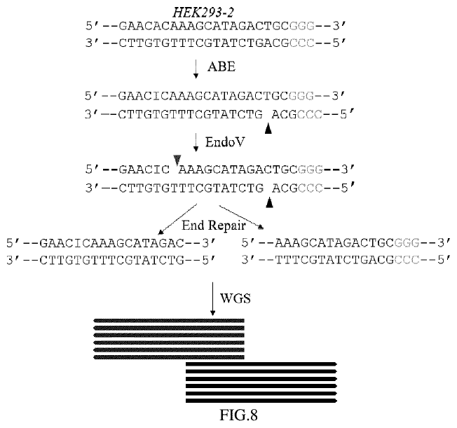

In order to further investigate the effectiveness and sensitivity of EndoV-seq. The pcDNA3.1-ABE7.10 vector (synthesized by Guangzhou Aiji Biotechnology Co., Ltd., SEQ ID NO 4) was co-transfected into 293T cells with a gRNA expression vector pUC19-SpCas9-gRNA (SEQ ID NO 5, constructed in laboratory) expressing HBG (or VEGFA3) gRNA, and cells were collected after 48 h. Genomic DNA was extracted using genomic DNA extraction kit (DNeasy Blood & Tissue Kit, Qiagen), the operation method was carried out completely according to the description. The target site and off-target site were then amplified by PCR using the primers in Table 2 and Table 3, and the PCR products were used for depth sequencing. As shown in FIG. 6, by depth sequencing, as for HBG, 6 sites in 18 off-target sites could be verified. As for VEGFA3, 3 sites in 22 off-target sites could be verified. Therefore, the total verification rate of EndoV-seq is 22.5% ( 9/40), indicating that the EndoV-seq can effectively detect the off-target effects of ABE. As for HBG gRNA, we find that the off-target efficiency of HBG-OT 9 sites in cells is 0.13%, very close to the detection limit 0.1%¹ of depth sequencing of PCR product (1. Tsai, S. Q. et al. CIRCLE-seq: a highly sensitive in vitro screen for genome-wide CRISPR-Cas9 nuclease off-targets. Nature methods 14, 607-614 (2017).). The results proof that the EndoV-seq has a very high sensitivity which can be at least 0.13%. The above results illustrate that the EndoV-seq can efficiently and sensitively detect the genome-wide off-target effects of ABE.

In order to further illustrate the beneficial effects of the present invention, the present invention provides the flowchart of the method for detecting the genome-wide off-target effects of ABE, as shown in FIG. 8.

As shown in FIG. 8, the TadA:TadA*:Cas9 fusion protein purified in vitro, gRNA, and genomic DNA were co-incubated in an embodiment of the present invention. In the reaction system, a DNA strand complementary to the gRNA were cut by the complex of TadA:TadA*:Cas9 and gRNA, while converting A on the non-complementary strand to I (Inosine). The genomic DNA containing the I base was then cut using Endonuclease V (EndoV), resulting in a DNA double-stranded break. Finally, the off-target effects of ABE is detected using whole-genome sequencing in conjunction with bioinformatics analysis.

Using the method for detecting the genome-wide off-target effects of ABE (Adenine base editor, ABE), adenine (A) at the target site can be efficiently substituted by guanine (G), which has a wide application prospect in gene editing for human disease treatment and disease model construction. But because the specificity of the CRISPR/Cas9 system is not high, the TadA:TadA*:Cas9 fusion protein may be targeted to the off-target sites which do not completely match gRNA, resulting in off-targets. The application of ABE is severely restricted. For this purpose, the first detection method EndoV-seq for detecting the genome-wide off-target effects of ABE, the off-target site of ABE can be detected in vitro by using the EndoV-seq, and verification is carried out in combination with the in-vivo experiment. It is envisioned that the EndoV-seq will have a wide application prospect in the field of gene editing, especially gene editing therapy field.

The sequence of SEQ ID No.4 and SEQ ID No.5 of the present invention is as follows: (the sequence of SEQ ID No.4 and SEQ ID No.5 is the sequence of the commercial plasmid vector, so that the following sequence is not provided in the following sequence list part):

(the sequence of pET42b-ABE7.10 vector)
SEQ ID NO. 3
GATATACCATGGGCAGCAGTCATCATCATCACCATCACTCGGAGGTTGAAT
TCTCCCACGAGTATTGGATGCGGCACGCTCTTACGTTAGCAAAACGCGCGTGGGACGA
GCGTGAAGTACCGGTAGGCGCCGTGTTAGTGCATAATAACCGGGTCATTGGTGAAGGA
TGGAATCGGCCGATCGGGAGACACGATCCGACAGCACATGCTGAGATTATGGCTTTAC
GGCAAGGAGGACTGGTTATGCAGAACTACCGGTTGATTGATGCTACACTGTACGTAAC
CTTAGAACCATGTGTGATGTGTGCTGGAGCCATGATACATTCCCGCATCGGAAGAGTG
GTTTTTGGGGCTCGTGATGCAAAAACTGGCGCCGCCGGAAGTCTTATGGACGTGTTAC
ATCATCCAGGCATGAATCATCGGGTCGAGATTACAGAGGGCATTTTGGCAGATGAATG
TGCTGCATTGCTTAGTGATTTCTTCCGCATGCGGAGACAGGAAATCAAAGCCCAAAAA
AAAGCTCAAAGTAGTACTGATAGTGGTGGATCCAGTGGAGGCTCGTCAGGCTCTGAAA
CGCCTGGCACATCAGAATCGGCAACGCCAGAGTCGTCAGGAGGTTCCTCAGGTGGATC
TTCGGAGGTCGAGTTTTCACATGAGTATTGGATGCGTCATGCCTTGACGTTGGCGAAAC
GGGCGCGCGATGAGCGTGAGGTGCCCGTGGGAGCGGTGTTGGTACTGAATAACCGGGT
TATAGGGGAAGGATGGAACCGGGCTATTGGGTTACACGACCCAACGGCGCACGCCGA
GATAATGGCACTGCGCCAAGGGGGCTTAGTTATGCAGAATTATCGCCTTATCGATGCT
ACACTGTATGTAACCTTTGAACCCTGCGTAATGTGTGCGGGGGCTATGATCCACTCGAG
AATAGGGCGCGTGGTATTCGGCGTACGCAACGCTAAAACCGGGGCTGCGGGCTCGTTG
ATGGATGTTCTGCACTACCCCGGAATGAATCACAGAGTAGAGATCACGGAGGGAATTT
TGGCCGACGAATGTGCAGCTTTACTGTGCTACTTTTTTCGGATGCCGCGGCAAGTCTTC
AACGCACAGAAGAAGGCTCAATCTTCCACTGACTCAGGTGGCTCGAGTGGTGGGAGTA
GCGGATCTGAGACGCCAGGCACATCAGAGAGTGCAACCCCCGAGTCATCGGGTGGGA
GTTCCGGCGGATCTGATAAGAAATACTCAATAGGCTTAGCTATCGGCACAAATAGCGT
CGGATGGGCGGTGATCACTGATGAATATAAGGTTCCGTCTAAAAAGTTCAAGGTTCTG
GGAAATACAGACCGCCACAGTATCAAAAAAAATCTTATAGGGGCTCTTTTATTTGACA
GTGGAGAGACAGCGGAAGCGACTCGTCTCAAACGGACAGCTCGTAGAAGGTATACAC
GTCGGAAGAATCGTATTTGTTATCTACAGGAGATTTTTTCAAATGAGATGGCGAAAGT
AGATGATAGTTTCTTTCATCGACTTGAAGAGTCTTTTTTGGTGGAAGAAGACAAGAAGC
ATGAACGTCATCCTATTTTTGGAAATATAGTAGATGAAGTTGCTTATCATGAGAAATAT
CCAACTATCTATCATCTGCGAAAAAAATTGGTAGATTCTACTGATAAAGCGGATTTGCG
CTTAATCTATTTGGCCTTAGCGCATATGATTAAGTTTCGTGGTCATTTTTTGATTGAGGG
AGATTTAAATCCTGATAATAGTGATGTGGACAAACTATTTATCCAGTTGGTACAAACCT
ACAATCAATTATTTGAAGAAAACCCTATTAACGCAAGTGGAGTAGATGCTAAAGCGAT
TCTTTCTGCACGATTGAGTAAATCAAGACGATTAGAAAATCTCATTGCTCAGCTCCCCG
GTGAGAAGAAAAATGGCTTATTTGGGAATCTCATTGCTTTGTCATTGGGTTTGACCCCT
AATTTTAAATCAAATTTTGATTTGGCAGAAGATGCTAAATTACAGCTTTCAAAAGATAC
TTACGATGATGATTTAGATAATTTATTGGCGCAAATTGGAGATCAATATGCTGATTTGT
TTTTGGCAGCTAAGAATTTATCAGATGCTATTTTACTTTCAGATATCCTAAGAGTAAAT
ACTGAAATAACTAAGGCTCCCCTATCAGCTTCAATGATTAAACGCTACGATGAACATC
ATCAAGACTTGACTCTTTTAAAAGCTTTAGTTCGACAACAACTTCCAGAAAAGTATAAA
GAAATCTTTTTTGATCAATCAAAAAACGGATATGCAGGTTATATTGATGGGGGAGCTA
GCCAAGAAGAATTTTATAAATTTATCAAACCAATTTTAGAAAAAATGGATGGTACTGA
GGAATTATTGGTGAAACTAAATCGTGAAGATTTGCTGCGCAAGCAACGGACCTTTGAC
AACGGCTCTATTCCCCATCAAATTCACTTGGGTGAGCTGCATGCTATTTTGAGAAGACA
AGAAGACTTTTATCCATTTTTAAAAGACAATCGTGAGAAGATTGAAAAAATCTTGACTT
TTCGAATTCCTTATTATGTTGGTCCATTGGCGCGTGGCAATAGTCGTTTTGCATGGATG
ACTCGGAAGTCTGAAGAAACAATTACCCCATGGAATTTTGAAGAAGTTGTCGATAAAG
GTGCTTCAGCTCAATCATTTATTGAACGCATGACAAACTTTGATAAAAATCTTCCAAAT
GAAAAAGTACTACCAAAACATAGTTTGCTTTATGAGTATTTTACGGTTTATAACGAATT
GACAAAGGTCAAATATGTTACTGAAGGAATGCGAAAACCAGCATTTCTTTCAGGTGAA
CAGAAGAAAGCCATTGTTGATTTACTCTTCAAAACAAATCGAAAAGTAACCGTTAAGC
AATTAAAAGAAGATTATTTCAAAAAAATAGAATGTTTTGATAGTGTTGAAATTTCAGG
AGTTGAAGATAGATTTAATGCTTCATTAGGTACCTACCATGATTTGCTAAAAATTATTA
AAGATAAAGATTTTTTGGATAATGAAGAAAATGAAGATATCTTAGAGGATATTGTTTT
AACATTGACCTTATTTGAAGATAGGGAGATGATTGAGGAAAGACTTAAAACATATGCT
CACCTCTTTGATGATAAGGTGATGAAACAGCTTAAACGTCGCCGTTATACTGGTTGGGG
ACGTTTGTCTCGAAAATTGATTAATGGTATTAGGGATAAGCAATCTGGCAAAACAATA
TTAGATTTTTTGAAATCAGATGGTTTTGCCAATCGCAATTTTATGCAGCTGATCCATGA
TGATAGTTTGACATTTAAAGAAGACATTCAAAAAGCACAAGTGTCTGGACAAGGCGAT
AGTTTACATGAACATATTGCAAATTTAGCTGGTAGCCCTGCTATTAAAAAAGGTATTTT
ACAGACTGTAAAAGTTGTTGATGAATTGGTCAAAGTAATGGGGCGGCATAAGCCAGAA
AATATCGTTATTGAAATGGCACGTGAAAATCAGACAACTCAAAAGGGCCAGAAAAATT
CGCGAGAGCGTATGAAACGAATCGAAGAAGGTATCAAAGAATTAGGAAGTCAGATTC
TTAAAGAGCATCCTGTTGAAAATACTCAATTGCAAAATGAAAAGCTCTATCTCTATTAT
CTCCAAAATGGAAGAGACATGTATGTGGACCAAGAATTAGATATTAATCGTTTAAGTG
ATTATGATGTCGATCACATTGTTCCACAAAGTTTCCTTAAAGACGATTCAATAGACAAT
AAGGTCTTAACGCGTTCTGATAAAAATCGTGGTAAATCGGATAACGTTCCAAGTGAAG
AAGTAGTCAAAAAGATGAAAAACTATTGGAGACAACTTCTAAACGCCAAGTTAATCAC
TCAACGTAAGTTTGATAATTTAACGAAAGCTGAACGTGGAGGTTTGAGTGAACTTGAT
AAAGCTGGTTTTATCAAACGCCAATTGGTTGAAACTCGCCAAATCACTAAGCATGTGG
CACAAATTTTGGATAGTCGCATGAATACTAAATACGATGAAAATGATAAACTTATTCG
AGAGGTTAAAGTGATTACCTTAAAATCTAAATTAGTTTCTGACTTCCGAAAAGATTTCC
AATTCTATAAAGTACGTGAGATTAACAATTACCATCATGCCCATGATGCGTATCTAAAT
GCCGTCGTTGGAACTGCTTTGATTAAGAAATATCCAAAACTTGAATCGGAGTTTGTCTA
TGGTGATTATAAAGTTTATGATGTTCGTAAAATGATTGCTAAGTCTGAGCAAGAAATA
GGCAAAGCAACCGCAAAATATTTCTTTTACTCTAATATCATGAACTTCTTCAAAACAGA
AATTACACTTGCAAATGGAGAGATTCGCAAACGCCCTCTAATCGAAACTAATGGGGAA
ACTGGAGAAATTGTCTGGGATAAAGGGCGAGATTTTGCCACAGTGCGCAAAGTATTGT
CCATGCCCCAAGTCAATATTGTCAAGAAAACAGAAGTACAGACAGGCGGATTCTCCAA
GGAGTCAATTTTACCAAAAAGAAATTCGGACAAGCTTATTGCTCGTAAAAAAGACTGG
GATCCAAAAAAATATGGTGGTTTTGATAGTCCAACGGTAGCTTATTCAGTCCTAGTTGT
TGCTAAGGTGGAAAAAGGGAAATCGAAGAAGTTAAAATCCGTTAAAGAGTTACTAGG
GATCACAATTATGGAAAGAAGTTCCTTTGAAAAAAATCCGATTGACTTTTTAGAAGCT
AAAGGATATAAGGAAGTTAAAAAAGACTTAATCATTAAACTACCTAAATATAGTCTTT
TTGAGTTAGAAAACGGTCGTAAACGGATGCTGGCTAGTGCCGGAGAATTACAAAAAGG
AAATGAGCTGGCTCTGCCAAGCAAATATGTGAATTTTTTATATTTAGCTAGTCATTATG
AAAAGTTGAAGGGTAGTCCAGAAGATAACGAACAAAAACAATTGTTTGTGGAGCAGC
ATAAGCATTATTTAGATGAGATTATTGAGCAAATCAGTGAATTTTCTAAGCGTGTTATT
TTAGCAGATGCCAATTTAGATAAAGTTCTTAGTGCATATAACAAACATAGAGACAAAC
CAATACGTGAACAAGCAGAAAATATTATTCATTTATTTACGTTGACGAATCTTGGAGCT
CCCGCTGCTTTTAAATATTTTGATACAACAATTGATCGTAAACGATATACGTCTACAAA
AGAAGTTTTAGATGCCACTCTTATCCATCAATCCATCACTGGTCTTTATGAAACACGCA
TTGATTTGAGTCAGCTAGGAGGTGACTCTGGCGGGTCTCCCAAGAAGAAGAGGAAAGT
CTAATAATTGATTAATACCTAGGCTGCTAAACAAAGCCCGAAAGGAAGCTGAGTTGGC
TGCTGCCACCGCTGAGCAATAACTAGCATAACCCCTTGGGGCCTCTAAACGGGTCTTG
AGGGGTTTTTTGCTGAAAGGAGGAACTATATCCGGATTGGCGAATGGGACGCGCCCTG
TAGCGGCGCATTAAGCGCGGCGGGTGTGGTGGTTACGCGCAGCGTGACCGCTACACTT
GCCAGCGCCCTAGCGCCCGCTCCTTTCGCTTTCTTCCCTTCCTTTCTCGCCACGTTCGCC
GGCTTTCCCCGTCAAGCTCTAAATCGGGGGCTCCCTTTAGGGTTCCGATTTAGTGCTTT
ACGGCACCTCGACCCCAAAAAACTTGATTAGGGTGATGGTTCACGTAGTGGGCCATCG
CCCTGATAGACGGTTTTTCGCCCTTTGACGTTGGAGTCCACGTTCTTTAATAGTGGACT
CTTGTTCCAAACTGGAACAACACTCAACCATATCTCGGTCTATTCTTTTGATTTATAAG
GGATTTTGCCGATTTCGGCCTATTGGTTAAAAAATGAGCTGATTTAACAAAAATTTAAC
GCGAATTTTAACAAAATATTAACGCTTACAATTTAGGTGGCACTTTTCGGGGAAATGTG
CGCGGAACCCCTATTTGTTTATTTTTCTAAATACATTCAAATATGTATCCGCTCATGAAT
TAATTCTTAGAAAAACTCATCGAGCATCAAATGAAACTGCAATTTATTCATATCAGGAT
TATCAATACCATATTTTTGAAAAAGCCGTTTCTGTAATGAAGGAGAAAACTCACCGAG
GCAGTTCCATAGGATGGCAAGATCCTGGTATCGGTCTGCGATTCCGACTCGTCCAACAT
CAATACAACCTATTAATTTCCCCTCGTCAAAAATAAGGTTATCAAGTGAGAAATCACC
ATGAGTGACGACTGAATCCGGTGAGAATGGCAAAAGTTTATGCATTTCTTTCCAGACTT
GTTCAACAGGCCAGCCATTACGCTCGTCATCAAAATCACTCGCATCAACCAAACCGTT
ATTCATTCGTGATTGCGCCTGAGCGAGACGAAATACGCGATCGCTGTTAAAAGGACAA
TTACAAACAGGAATCGAATGCAACCGGCGCAGGAACACTGCCAGCGCATCAACAATAT
TTTCACCTGAATCAGGATATTCTTCTAATACCTGGAATGCTGTTTTCCCGGGGATCGCA
GTGGTGAGTAACCATGCATCATCAGGAGTACGGATAAAATGCTTGATGGTCGGAAGAG
GCATAAATTCCGTCAGCCAGTTTAGTCTGACCATCTCATCTGTAACATCATTGGCAACG
CTACCTTTGCCATGTTTCAGAAACAACTCTGGCGCATCGGGCTTCCCATACAATCGATA
GATTGTCGCACCTGATTGCCCGACATTATCGCGAGCCCATTTATACCCATATAAATCAG
CATCCATGTTGGAATTTAATCGCGGCCTAGAGCAAGACGTTTCCCGTTGAATATGGCTC
ATAACACCCCTTGTATTACTGTTTATGTAAGCAGACAGTTTTATTGTTCATGACCAAAA
TCCCTTAACGTGAGTTTTCGTTCCACTGAGCGTCAGACCCCGTAGAAAAGATCAAAGG
ATCTTCTTGAGATCCTTTTTTTCTGCGCGTAATCTGCTGATTGCAAACAAAAAAACCAC
CGCTACCAGCGGTGGTTTGTTTGCCGGATCAAGAGCTACCAACTCTTTTTCCGAAGGTA
ACTGGCTTCAGCAGAGCGCAGATACCAAATACTGTCCTTCTAGTGTAGCCGTAGTTAG
GCCACCACTTCAAGAACTCTGTAGCACCGCCTACATACCTCGCTCTGCTAATCATGTTA
CCAGTGGCTGCTGCCAGTGGCGATAAGTCGTGTCTTACCGGGTTGGACTCAAGACGAT
AGTTACCGGATAAGGCGCAGCGGTCGGGCTGAACGGGGGGTTCGTGCACACAGCCAA
GCTTGGAGCGAACGACCTACACCGAACTGAGATACCTACAGCGTGAGCTATGAGAAAG
CGCCACGCTTCCCGAAGGGAGAAAGGCGGACAGGTATCCGGTAAGCGGCAGGGTCGG
AACAGGAGAGCGCACGAGGGAGCTTCCAGGGGGAAACGCCTGGTATCTTTATAGTCCT
GTCGGGTTTCGCCACCTCTGACTTGAGCGTCGATTTTTGTGATGCTCGTCAGGGGGGCG
GAGCCTATGGAAAAACGCCAGCAACGCGGCCTTTTTACGGTTCCTGGCCTTTTGCTGGC
CTTTTGCTCACATGTTCTTTCCTGCGTTATCCCCTGATTCTGTGGATAACCGTATTACCG
CCTTTGAGTGAGCTGATACCGCTCGCCGCAGCCGAACGACCGAGCGCAGCGAGTCAGT
GAGCGAGGAAGCGGAAGAGCGCCTGATGCGGTATTTTCTCCTTACGCATCTGTGCGGT
ATTTCACACCGCAATGGTGCACTCTCAGTACAATCTGCTCTGATGCCGCATAGTTAAGC
CAGTATACACTCCGCTATCGCTACGTGACTGGGTCATGGCTGCGCCCCGACACCCGCCA
ACACCCGCTGACGCGCCCTGACGGGCTTGTCTGCTCCCGGCATCCGCTTACAGACAAG
CTGTGACCGTCTCCGGGAGCTGCATGTGTCAGAGGTTTTCACCGTCATCACCGAAACGC
GCGAGGCAGCTGCGGTAAAGCTCATAAGCGTGGTCGTGAAGCGATTCACAGATGTCTG
CCTGTTCATCCGCGTCCAGCTCGTTGAGTTTCTCCAGAAGCGTTAATGTCTGGCTTCTG
ATAAAGCGGGCCATGTTAAGGGCGGTTTTTTCCTGTTTGGTCACTGATGCCTCCGTGTA
AGGGGGATTTCTGTTCATGGGGGTAATGATACCGATGAAACGAGAGAGGATGCTCACG
ATACGGGTTACTGATGATGAACATGCCCGGTTACTGGAACGTTGTGAGGGTAAACAAC
TGGCGGTATGGATGCGGCGGGACCAGAGAAAAATCACTCAGGGTCAATGCCAGCGCTT
CGTTAATACAGATGTAGGTGTTCCACAGGGTAGCCAGCAGCATCCTGCGATGCAGATC
CGGAACATAATGGTGCAGGGCGCTGACTTCCGCGTTTCCAGACTTTACGAAACACGGA
AACCGAAGACCATTCATGTTGTTGCTCAGGTCGCAGACGTTTTGCAGCAGCAGTCGCTT
CACGTTCGCTCGCGTATCGGTGATTCATTCTGCTAACCAGTAAGGCAACCCCGCCAGCC
TAGCCGGGTCCTCAACGACAGGAGCACGATCATGCGCACCCGTGGGGCCGCCATGCCG
GCGATAATGGCCTGCTTCTCGCCGAAACGTTTGGTGGCGGGACCAGTGACGAAGGCTT
GAGCGAGGGAGTGCAAGATTCCGAATACCGCAAGCGACAGGCCGATCATCGTCGCGCT
CCAGCGAAAGCGGTCCTCGCCGAAAATGACCCAGAGCGCTGCCGGCACCTGTCCTACG
AGTTGCATGATAAAGAAGACAGTCATAAGTGCGGCGACGATAGTCATGCCCCGCGCCC
ACCGGAAGGAGCTGACTGGGTTGAAGGCTCTCAAGGGCATCGGTCGAGATCCCGGTGC
CTAATGAGTGAGCTAACTTACATTAATTGCGTTGCGCTCACTGCCAGCTTTCCAGTCGG
GAAACCTGTCGTGCCAGCTGCATTAATGAATCGGCCAACGCGCGGGGAGAGGCGGTTT
GCGTATTGGGCGCCAGGGTGGTTTTTCTTTTCACCAGTGAGACGGGCAACAGCTGATTG
CCCTTCACCGCCTGGCCCTGAGAGAGTTGCAGCAAGCGGTCCACGCTGGTTTGCCCCA
GCAGGCGAAAATCCTGTTTGATGGTGGTTAACGGCGGGATATAACATGAGCTGTCTTC
GGTATCGTCGTATCCCACTACCGAGATATCCGCACCAACGCGCAGCCCGGACTCGGTA
ATGGCGCGCATTGCGCCCAGCGCCATCTGATCGTTGGCAACCAGCATCGCAGTGGGAA
CGATGCCCTCATTCAGCATTTGCATGGTTTGTTGAAAACCGGACATGGCACTCCAGTCG
CCTTCCCGTTCCGCTATCGGCTGAATTTGATTGCGAGTGAGATATTTATGCCAGCCAGC
CAGACGCAGACGCGCCGAGACAGAACTTAATGGGCCCGCTAACAGCGCGATTTGCTGG
TGACCCAATGCGACCAGATGCTCCACGCCCAGTCGCGTACCGTCTTCATGGGAGAAAA
TAATACTGTTGATGGGTGTCTGGTCAGAGACATCAAGAAATAACGCCGGAACATTAGT
GCAGGCAGCTTCCACAGCAATGGCATCCTGGTCATCCAGCGGATAGTTAATGATCAGC
CCACTGACGCGTTGCGCGAGAAGATTGTGCACCGCCGCTTTACAGGCTTCGACGCCGC
TTCGTTCTACCATCGACACCACCACGCTGGCACCCAGTTGATCGGCGCGAGATTTAATC
GCCGCGACAATTTGCGACGGCGCGTGCAGGGCCAGACTGGAGGTGGCAACGCCAATC
AGCAACAACTGTTTGCCCGCCAGTTGTTGTGCCACGCGGTTGGGAATGTAATTCAGCTC
CGCCATCGCCGCTTCCACTTTTTCCCGCGTTTTCGCAGAAACGTGGCTGGCATGGTTCA
CCACGCGGGAAACGGTCTGATAAGAGACACCGGCATACTCTGCGACATCGTATAACGT
TACTGGTTTCACATTCACCACCCTGAATTGACTCTCTTCCGGGCGCTATCATGCCATAC
CGCGAAAGGTTTTGCGCCATTCGATGGTGTCCGGGATCTCGACGCTCTCCCTTATGCGA
CTCCTGCATTAGGAAGCAGCCCAGTAGTAGGTTGAGGCCGTTGAGCACCGCCGCCGCA
AGGAATGGTGCATGCAAGGAGATGGCGCCCAACAGTCCCCCGGCCACGGGGCCTGCC
ACCATACCCACGCCGAAACAAGCGCTCATGAGCCCAAAGTGGCGAGCCCGATCTTCCC
CATCGGTGATGTCGGCGATATAGGCGCCAGCAACCGCACCTGTGGCGCCGGTGATGCC
GGCCACGATGCGTCCGGCGTAGAGGATCGAGATCTCGATCCCGCGAAATTAATACGAC
TCACTATAGGGGAATTGTGAGCGGATAACAATTCCCCTCTAGAAATAATTTTGTTTAAC
TTTAAGAAGGA
(the sequence of pcDNA3.1-ABE7.10 vector)
SEQ ID NO. 4
AGCTTAAGTTTAAACCGCTGATCAGCCTCGACTGTGCCTTCTAGTTGCCAGCCATCTGT
TGTTTGCCCCTCCCCCGTGCCTTCCTTGACCCTGGAAGGTGCCACTCCCACTGTCCTTTC
CTAATAAAATGAGGAAATTGCATCGCATTGTCTGAGTAGGTGTCATTCTATTCTGGGGG
GTGGGGTGGGGCAGGACAGCAAGGGGGAGGATTGGGAAGACAATAGCAGGCATGCTG
GGGATGCGGTGGGCTCTATGGCTTCTGAGGCGGAAAGAACCAGCTGGGGCTCTAGGGG
GTATCCCCACGCGCCCTGTAGCGGCGCATTAAGCGCGGCGGGTGTGGTGGTTACGCGC
AGCGTGACCGCTACACTTGCCAGCGCCCTAGCGCCCGCTCCTTTCGCTTTCTTCCCTTCC
TTTCTCGCCACGTTCGCCGGCTTTCCCCGTCAAGCTCTAAATCGGGGGCTCCCTTTAGG
GTTCCGATTTAGTGCTTTACGGCACCTCGACCCCAAAAAACTTGATTAGGGTGATGGTT
CACGTAGTGGGCCATCGCCCTGATAGACGGTTTTTCGCCCTTTGACGTTGGAGTCCACG
TTCTTTAATAGTGGACTCTTGTTCCAAACTGGAACAACACTCAACCCTATCTCGGTCTA
TTCTTTTGATTTATAAGGGATTTTGCCGATTTCGGCCTATTGGTTAAAAAATGAGCTGA
TTTAACAAAAATTTAACGCGAATTAATTCTGTGGAATGTGTGTCAGTTAGGGTGTGGAA
AGTCCCCAGGCTCCCCAGCAGGCAGAAGTATGCAAAGCATGCATCTCAATTAGTCAGC
AACCAGGTGTGGAAAGTCCCCAGGCTCCCCAGCAGGCAGAAGTATGCAAAGCATGCAT
CTCAATTAGTCAGCAACCATAGTCCCGCCCCTAACTCCGCCCATCCCGCCCCTAACTCC
GCCCAGTTCCGCCCATTCTCCGCCCCATGGCTGACTAATTTTTTTTATTTATGCAGAGGC
CGAGGCCGCCTCTGCCTCTGAGCTATTCCAGAAGTAGTGAGGAGGCTTTTTTGGAGGCC
TAGGCTTTTGCAAAAAGCTCCCGGGAGCTTGTATATCCATTTTCGGATCTGATCAAGAG
ACAGGATGAGGATCGTTTCGCATGATTGAACAAGATGGATTGCACGCAGGTTCTCCGG
CCGCTTGGGTGGAGAGGCTATTCGGCTATGACTGGGCACAACAGACAATCGGCTGCTC
TGATGCCGCCGTGTTCCGGCTGTCAGCGCAGGGGCGCCCGGTTCTTTTTGTCAAGACCG
ACCTGTCCGGTGCCCTGAATGAACTGCAGGACGAGGCAGCGCGGCTATCGTGGCTGGC
CACGACGGGCGTTCCTTGCGCAGCTGTGCTCGACGTTGTCACTGAAGCGGGAAGGGAC
TGGCTGCTATTGGGCGAAGTGCCGGGGCAGGATCTCCTGTCATCTCACCTTGCTCCTGC
CGAGAAAGTATCCATCATGGCTGATGCAATGCGGCGGCTGCATACGCTTGATCCGGCT
ACCTGCCCATTCGACCACCAAGCGAAACATCGCATCGAGCGAGCACGTACTCGGATGG
AAGCCGGTCTTGTCGATCAGGATGATCTGGACGAAGAGCATCAGGGGCTCGCGCCAGC
CGAACTGTTCGCCAGGCTCAAGGCGCGCATGCCCGACGGCGAGGATCTCGTCGTGACC
CATGGCGATGCCTGCTTGCCGAATATCATGGTGGAAAATGGCCGCTTTTCTGGATTCAT
CGACTGTGGCCGGCTGGGTGTGGCGGACCGCTATCAGGACATAGCGTTGGCTACCCGT
GATATTGCTGAAGAGCTTGGCGGCGAATGGGCTGACCGCTTCCTCGTGCTTTACGGTAT
CGCCGCTCCCGATTCGCAGCGCATCGCCTTCTATCGCCTTCTTGACGAGTTCTTCTGAG
CGGGACTCTGGGGTTCGAAATGACCGACCAAGCGACGCCCAACCTGCCATCACGAGAT
TTCGATTCCACCGCCGCCTTCTATGAAAGGTTGGGCTTCGGAATCGTTTTCCGGGACGC
CGGCTGGATGATCCTCCAGCGCGGGGATCTCATGCTGGAGTTCTTCGCCCACCCCAACT
TGTTTATTGCAGCTTATAATGGTTACAAATAAAGCAATAGCATCACAAATTTCACAAAT
AAAGCATTTTTTTCACTGCATTCTAGTTGTGGTTTGTCCAAACTCATCAATGTATCTTAT
CATGTCTGTATACCGTCGACCTCTAGCTAGAGCTTGGCGTAATCATGGTCATAGCTGTT
TCCTGTGTGAAATTGTTATCCGCTCACAATTCCACACAACATACGAGCCGGAAGCATA
AAGTGTAAAGCCTGGGGTGCCTAATGAGTGAGCTAACTCACATTAATTGCGTTGCGCT
CACTGCCCGCTTTCCAGTCGGGAAACCTGTCGTGCCAGCTGCATTAATGAATCGGCCAA
CGCGCGGGGAGAGGCGGTTTGCGTATTGGGCGCTCTTCCGCTTCCTCGCTCACTGACTC
GCTGCGCTCGGTCGTTCGGCTGCGGCGAGCGGTATCAGCTCACTCAAAGGCGGTAATA
CGGTTATCCACAGAATCAGGGGATAACGCAGGAAAGAACATGTGAGCAAAAGGCCAG
CAAAAGGCCAGGAACCGTAAAAAGGCCGCGTTGCTGGCGTTTTTCCATAGGCTCCGCC
CCCCTGACGAGCATCACAAAAATCGACGCTCAAGTCAGAGGTGGCGAAACCCGACAG
GACTATAAAGATACCAGGCGTTTCCCCCTGGAAGCTCCCTCGTGCGCTCTCCTGTTCCG
ACCCTGCCGCTTACCGGATACCTGTCCGCCTTTCTCCCTTCGGGAAGCGTGGCGCTTTC
TCATAGCTCACGCTGTAGGTATCTCAGTTCGGTGTAGGTCGTTCGCTCCAAGCTGGGCT
GTGTGCACGAACCCCCCGTTCAGCCCGACCGCTGCGCCTTATCCGGTAACTATCGTCTT
GAGTCCAACCCGGTAAGACACGACTTATCGCCACTGGCAGCAGCCACTGGTAACAGGA
TTAGCAGAGCGAGGTATGTAGGCGGTGCTACAGAGTTCTTGAAGTGGTGGCCTAACTA
CGGCTACACTAGAAGAACAGTATTTGGTATCTGCGCTCTGCTGAAGCCAGTTACCTTCG
GAAAAAGAGTTGGTAGCTCTTGATCCGGCAAACAAACCACCGCTGGTAGCGGTTTTTT
TGTTTGCAAGCAGCAGATTACGCGCAGAAAAAAAGGATCTCAAGAAGATCCTTTGATC
TTTTCTACGGGGTCTGACGCTCAGTGGAACGAAAACTCACGTTAAGGGATTTTGGTCAT
GAGATTATCAAAAAGGATCTTCACCTAGATCCTTTTAAATTAAAAATGAAGTTTTAAAT
CAATCTAAAGTATATATGAGTAAACTTGGTCTGACAGTTACCAATGCTTAATCAGTGAG
GCACCTATCTCAGCGATCTGTCTATTTCGTTCATCCATAGTTGCCTGACTCCCCGTCGTG
TAGATAACTACGATACGGGAGGGCTTACCATCTGGCCCCAGTGCTGCAATGATACCGC
GAGACCCACGCTCACCGGCTCCAGATTTATCAGCAATAAACCAGCCAGCCGGAAGGGC
CGAGCGCAGAAGTGGTCCTGCAACTTTATCCGCCTCCATCCAGTCTATTAATTGTTGCC
GGGAAGCTAGAGTAAGTAGTTCGCCAGTTAATAGTTTGCGCAACGTTGTTGCCATTGCT
ACAGGCATCGTGGTGTCACGCTCGTCGTTTGGTATGGCTTCATTCAGCTCCGGTTCCCA
ACGATCAAGGCGAGTTACATGATCCCCCATGTTGTGCAAAAAAGCGGTTAGCTCCTTC
GGTCCTCCGATCGTTGTCAGAAGTAAGTTGGCCGCAGTGTTATCACTCATGGTTATGGC
AGCACTGCATAATTCTCTTACTGTCATGCCATCCGTAAGATGCTTTTCTGTGACTGGTG
AGTACTCAACCAAGTCATTCTGAGAATAGTGTATGCGGCGACCGAGTTGCTCTTGCCCG
GCGTCAATACGGGATAATACCGCGCCACATAGCAGAACTTTAAAAGTGCTCATCATTG
GAAAACGTTCTTCGGGGCGAAAACTCTCAAGGATCTTACCGCTGTTGAGATCCAGTTC
GATGTAACCCACTCGTGCACCCAACTGATCTTCAGCATCTTTTACTTTCACCAGCGTTTC
TGGGTGAGCAAAAACAGGAAGGCAAAATGCCGCAAAAAAGGGAATAAGGGCGACAC
GGAAATGTTGAATACTCATACTCTTCCTTTTTCAATATTATTGAAGCATTTATCAGGGTT
ATTGTCTCATGAGCGGATACATATTTGAATGTATTTAGAAAAATAAACAAATAGGGGT
TCCGCGCACATTTCCCCGAAAAGTGCCACCTGACGTCGACGGATCGGGAGATCTCCCG
ATCCCCTATGGTGCACTCTCAGTACAATCTGCTCTGATGCCGCATAGTTAAGCCAGTAT
CTGCTCCCTGCTTGTGTGTTGGAGGTCGCTGAGTAGTGCGCGAGCAAAATTTAAGCTAC
AACAAGGCAAGGCTTGACCGACAATTGCATGAAGAATCTGCTTAGGGTTAGGCGTTTT
GCGCTGCTTCGCGATGTACGGGCCAGATATACGCGTTGACATTGATTATTGACTAGTTA
TTAATAGTAATCAATTACGGGGTCATTAGTTCATAGCCCATATATGGAGTTCCGCGTTA
CATAACTTACGGTAAATGGCCCGCCTGGCTGACCGCCCAACGACCCCCGCCCATTGAC
GTCAATAATGACGTATGTTCCCATAGTAACGCCAATAGGGACTTTCCATTGACGTCAAT
GGGTGGAGTATTTACGGTAAACTGCCCACTTGGCAGTACATCAAGTGTATCATATGCC
AAGTACGCCCCCTATTGACGTCAATGACGGTAAATGGCCCGCCTGGCATTATGCCCAG
TACATGACCTTATGGGACTTTCCTACTTGGCAGTACATCTACGTATTAGTCATCGCTATT
ACCATGGTGATGCGGTTTTGGCAGTACATCAATGGGCGTGGATAGCGGTTTGACTCAC
GGGGATTTCCAAGTCTCCACCCCATTGACGTCAATGGGAGTTTGTTTTGGCACCAAAAT
CAACGGGACTTTCCAAAATGTCGTAACAACTCCGCCCCATTGACGCAAATGGGCGGTA
GGCGTGTACGGTGGGAGGTCTATATAAGCAGAGCTCTCTGGCTAACTAGAGAACCCAC
TGCTTACTGGCTTATCGAAATTAATACGACTCACTATAGGGAGACCCAAGCTGGCTAG
CGTTTAAACGGGCCCTCTAGACTCGAGCGGCCGCCATGTCCGAAGTCGAGTTTTCCCAT
GAGTACTGGATGAGACACGCATTGACTCTCGCAAAGAGGGCTTGGGATGAACGCGAG
GTGCCCGTGGGGGCAGTACTCGTGCATAACAATCGCGTAATCGGCGAAGGTTGGAATA
GGCCGATCGGACGCCACGACCCCACTGCACATGCGGAAATCATGGCCCTTCGACAGGG
AGGGCTTGTGATGCAGAATTATCGACTTATCGATGCGACGCTGTACGTCACGCTTGAAC
CTTGCGTAATGTGCGCGGGAGCTATGATTCACTCCCGCATTGGACGAGTTGTATTCGGT
GCCCGCGACGCCAAGACGGGTGCCGCAGGTTCACTGATGGACGTGCTGCATCACCCAG
GCATGAACCACCGGGTAGAAATCACAGAAGGCATATTGGCGGACGAATGTGCGGCGC
TGTTGTCCGACTTTTTTCGCATGCGGAGGCAGGAGATCAAGGCCCAGAAAAAAGCACA
ATCCTCTACTGACTCTGGTGGTTCTTCTGGTGGTTCTAGCGGCAGCGAGACTCCCGGGA
CCTCAGAGTCCGCCACACCCGAAAGTTCTGGTGGTTCTTCTGGTGGTTCTTCCGAGGTC
GAATTTTCACATGAGTATTGGATGCGACACGCCTTGACGCTCGCCAAAAGGGCGAGGG
ACGAACGGGAAGTTCCCGTAGGCGCCGTCCTTGTACTGAATAATCGAGTTATTGGCGA
AGGTTGGAACAGGGCCATAGGACTGCATGATCCAACAGCCCATGCAGAAATCATGGCG
CTCCGGCAGGGTGGCCTTGTCATGCAAAATTATAGGCTGATCGACGCGACGTTGTACG
TCACCTTCGAACCTTGCGTTATGTGTGCAGGCGCTATGATACATTCAAGAATTGGGCGA
GTCGTGTTTGGGGTCAGGAACGCAAAGACTGGTGCAGCCGGTTCCCTTATGGATGTGC
TCCACTACCCAGGAATGAATCATCGGGTCGAGATTACAGAGGGGATACTGGCTGACGA
ATGCGCCGCCCTCCTGTGCTACTTCTTTCGGATGCCCAGGCAGGTGTTTAACGCACAGA
AGAAAGCTCAAAGCAGTACCGACTCTGGGGGCTCTAGTGGAGGCTCCAGCGGTTCTGA
GACCCCCGGCACTAGTGAATCTGCCACTCCCGAATCATCCGGGGGATCTTCAGGGGGA
TCTGATAAAAAGTATTCTATTGGTTTAGCCATCGGCACTAATTCCGTTGGATGGGCTGT
CATAACCGATGAATACAAAGTACCTTCAAAGAAATTTAAGGTGTTGGGGAACACAGAC
CGTCATTCGATTAAAAAGAATCTTATCGGTGCCCTCCTATTCGATAGTGGCGAAACGGC
AGAGGCGACTCGCCTGAAACGAACCGCTCGGAGAAGGTATACACGTCGCAAGAACCG
AATATGTTACTTACAAGAAATTTTTAGCAATGAGATGGCCAAAGTTGACGATTCTTTCT
TTCACCGTTTGGAAGAGTCCTTCCTTGTCGAAGAGGACAAGAAACATGAACGGCACCC
CATCTTTGGAAACATAGTAGATGAGGTGGCATATCATGAAAAGTACCCAACGATTTAT
CACCTCAGAAAAAAGCTAGTTGACTCAACTGATAAAGCGGACCTGAGGTTAATCTACT
TGGCTCTTGCCCATATGATAAAGTTCCGTGGGCACTTTCTCATTGAGGGTGATCTAAAT
CCGGACAACTCGGATGTCGACAAACTGTTCATCCAGTTAGTACAAACCTATAATCAGTT
GTTTGAAGAGAACCCTATAAATGCAAGTGGCGTGGATGCGAAGGCTATTCTTAGCGCC
CGCCTCTCTAAATCCCGACGGCTAGAAAACCTGATCGCACAATTACCCGGAGAGAAGA
AAAATGGGTTGTTCGGTAACCTTATAGCGCTCTCACTAGGCCTGACACCAAATTTTAAG
TCGAACTTCGACTTAGCTGAAGATGCCAAATTGCAGCTTAGTAAGGACACGTACGATG
ACGATCTCGACAATCTACTGGCACAAATTGGAGATCAGTATGCGGACTTATTTTTGGCT
GCCAAAAACCTTAGCGATGCAATCCTCCTATCTGACATACTGAGAGTTAATACTGAGA
TTACCAAGGCGCCGTTATCCGCTTCAATGATCAAAAGGTACGATGAACATCACCAAGA
CTTGACACTTCTCAAGGCCCTAGTCCGTCAGCAACTGCCTGAGAAATATAAGGAAATA
TTCTTTGATCAGTCGAAAAACGGGTACGCAGGTTATATTGACGGCGGAGCGAGTCAAG
AGGAATTCTACAAGTTTATCAAACCCATATTAGAGAAGATGGATGGGACGGAAGAGTT
GCTTGTAAAACTCAATCGCGAAGATCTACTGCGAAAGCAGCGGACTTTCGACAACGGT
AGCATTCCACATCAAATCCACTTAGGCGAATTGCATGCTATACTTAGAAGGCAGGAGG
ATTTTTATCCGTTCCTCAAAGACAATCGTGAAAAGATTGAGAAAATCCTAACCTTTCGC
ATACCTTACTATGTGGGACCCCTGGCCCGAGGGAACTCTCGGTTCGCATGGATGACAA
GAAAGTCCGAAGAAACGATTACTCCCTGGAATTTTGAGGAAGTTGTCGATAAAGGTGC
GTCAGCTCAATCGTTCATCGAGAGGATGACCAACTTTGACAAGAATTTACCGAACGAA
AAAGTATTGCCTAAGCACAGTTTACTTTACGAGTATTTCACAGTGTACAATGAACTCAC
GAAAGTTAAGTATGTCACTGAGGGCATGCGTAAACCCGCCTTTCTAAGCGGAGAACAG
AAGAAAGCAATAGTAGATCTGTTATTCAAGACCAACCGCAAAGTGACAGTTAAGCAAT
TGAAAGAGGACTACTTTAAGAAAATTGAATGCTTCGATTCTGTCGAGATCTCCGGGGT
AGAAGATCGATTTAATGCGTCACTTGGTACGTATCATGACCTCCTAAAGATAATTAAA
GATAAGGACTTCCTGGATAACGAAGAGAATGAAGATATCTTAGAAGATATAGTGTTGA
CTCTTACCCTCTTTGAAGATCGGGAAATGATTGAGGAAAGACTAAAAACATACGCTCA
CCTGTTCGACGATAAGGTTATGAAACAGTTAAAGAGGCGTCGCTATACGGGCTGGGGA
CGCTTGTCGCGGAAACTTATCAACGGGATAAGAGACAAGCAAAGTGGTAAAACTATTC
TCGATTTTCTAAAGAGCGACGGCTTCGCCAATAGGAACTTTATGCAGCTGATCCATGAT
GACTCTTTAACCTTCAAAGAGGATATACAAAAGGCACAGGTTTCCGGACAAGGGGACT
CATTGCACGAACATATTGCGAATCTTGCTGGTTCGCCAGCCATCAAAAAGGGCATACT
CCAGACAGTCAAAGTAGTGGATGAGCTAGTTAAGGTCATGGGACGTCACAAACCGGA
AAACATTGTAATCGAGATGGCACGCGAAAATCAAACGACTCAGAAGGGGCAAAAAAA
CAGTCGAGAGCGGATGAAGAGAATAGAAGAGGGTATTAAAGAACTGGGCAGCCAGAT
CTTAAAGGAGCATCCTGTGGAAAATACCCAATTGCAGAACGAGAAACTTTACCTCTAT
TACCTACAAAATGGAAGGGACATGTATGTTGATCAGGAACTGGACATAAACCGTTTAT
CTGATTACGACGTCGATCACATTGTACCCCAATCCTTTTTGAAGGACGATTCAATCGAC
AATAAAGTGCTTACACGCTCGGATAAGAACCGAGGGAAAAGTGACAATGTTCCAAGC
GAGGAAGTCGTAAAGAAAATGAAGAACTATTGGCGGCAGCTCCTAAATGCGAAACTG
ATAACGCAAAGAAAGTTCGATAACTTAACTAAAGCTGAGAGGGGTGGCTTGTCTGAAC
TTGACAAGGCCGGATTTATTAAACGTCAGCTCGTGGAAACCCGCCAGATCACAAAGCA
TGTTGCCCAGATACTAGATTCCCGAATGAATACGAAATACGACGAGAACGATAAGCTG
ATTCGGGAAGTCAAAGTAATCACTTTAAAGTCAAAATTGGTGTCGGACTTCAGAAAGG
ATTTTCAATTCTATAAAGTTAGGGAGATAAATAACTACCACCATGCGCACGACGCTTAT
CTTAATGCCGTCGTAGGGACCGCACTCATTAAGAAATACCCGAAGCTAGAAAGTGAGT
TTGTGTATGGTGATTACAAAGTTTATGACGTCCGTAAGATGATCGCGAAAAGCGAACA
GGAGATAGGCAAGGCTACAGCCAAATACTTCTTTTATTCTAACATTATGAATTTCTTTA
AGACGGAAATCACTCTGGCAAACGGAGAGATACGCAAACGACCTTTAATTGAAACCA
ATGGGGAGACAGGTGAAATCGTATGGGATAAGGGCCGGGACTTCGCGACGGTGAGAA
AAGTTTTGTCCATGCCCCAAGTCAACATAGTAAAGAAAACTGAGGTGCAGACCGGAGG
GTTTTCAAAGGAATCGATTCTTCCAAAAAGGAATAGTGATAAGCTCATCGCTCGTAAA
AAGGACTGGGACCCGAAAAAGTACGGTGGCTTCGATAGCCCTACAGTTGCCTATTCTG
TCCTAGTAGTGGCAAAAGTTGAGAAGGGAAAATCCAAGAAACTGAAGTCAGTCAAAG
AATTATTGGGGATAACGATTATGGAGCGCTCGTCTTTTGAAAAGAACCCCATCGACTTC
CTTGAGGCGAAAGGTTACAAGGAAGTAAAAAAGGATCTCATAATTAAACTACCAAAGT
ATAGTCTGTTTGAGTTAGAAAATGGCCGAAAACGGATGTTGGCTAGCGCCGGAGAGCT
TCAAAAGGGGAACGAACTCGCACTACCGTCTAAATACGTGAATTTCCTGTATTTAGCGT
CCCATTACGAGAAGTTGAAAGGTTCACCTGAAGATAACGAACAGAAGCAACTTTTTGT
TGAGCAGCACAAACATTATCTCGACGAAATCATAGAGCAAATTTCGGAATTCAGTAAG
AGAGTCATCCTAGCTGATGCCAATCTGGACAAAGTATTAAGCGCATACAACAAGCACA
GGGATAAACCCATACGTGAGCAGGCGGAAAATATTATCCATTTGTTTACTCTTACCAAC
CTCGGCGCTCCAGCCGCATTCAAGTATTTTGACACAACGATAGATCGCAAACGATACA
CTTCTACCAAGGAGGTGCTAGACGCGACACTGATTCACCAATCCATCACGGGATTATA
TGAAACTCGGATAGATTTGTCACAGCTTGGGGGTGACTCTGGTGGTTCTCCCAAGAAG
AAGAGGAAAGTCTAAA
(the sequence of pUC19-SpCas9-gRNA vector)
SEQ ID NO. 5
TCGCGCGTTTCGGTGATGACGGTGAAAACCTCTGACACATGCAGCTCCCGGAGACGGT
CACAGCTTGTCTGTAAGCGGATGCCGGGAGCAGACAAGCCCGTCAGGGCGCGTCAGCG
GGTGTTGGCGGGTGTCGGGGCTGGCTTAACTATGCGGCATCAGAGCAGATTGTACTGA
GAGTGCACCATATGCGGTGTGAAATACCGCACAGATGCGTAAGGAGAAAATACCGCAT
CAGGCGCCATTCGCCATTCAGGCTGCGCAACTGTTGGGAAGGGCGATCGGTGCGGGCC
TCTTCGCTATTACGCCAGCTGGCGAAAGGGGGATGTGCTGCAAGGCGATTAAGTTGGG
TAACGCCAGGGTTTTCCCAGTCACGACGTTGTAAAACGACGGCCAGTGAATTCGAGGG
CCTATTTCCCATGATTCCTTCATATTTGCATATACGATACAAGGCTGTTAGAGAGATAA
TTGGAATTAATTTGACTGTAAACACAAAGATATTAGTACAAAATACGTGACGTAGAAA
GTAATAATTTCTTGGGTAGTTTGCAGTTTTAAAATTATGTTTTAAAATGGACTATCATAT
GCTTACCGTAACTTGAAAGTATTTCGATTTCTTGGCTTTATATATCTTGTGGAAAGGAC
GAAACACCGGGTCTTCGAGAAGACCTGTTTTAGAGCTAGAAATAGCAAGTTAAAATAA
GGCTAGTCCGTTATCAACTTGAAAAAGTGGCACCGAGTCGGTGCTTTTTTCTAGAGTCG
ACCTGCAGGCATGCAAGCTTGGCGTAATCATGGTCATAGCTGTTTCCTGTGTGAAATTG
TTATCCGCTCACAATTCCACACAACATACGAGCCGGAAGCATAAAGTGTAAAGCCTGG
GGTGCCTAATGAGTGAGCTAACTCACATTAATTGCGTTGCGCTCACTGCCCGCTTTCCA
GTCGGGAAACCTGTCGTGCCAGCTGCATTAATGAATCGGCCAACGCGCGGGGAGAGGC
GGTTTGCGTATTGGGCGCTCTTCCGCTTCCTCGCTCACTGACTCGCTGCGCTCGGTCGTT
CGGCTGCGGCGAGCGGTATCAGCTCACTCAAAGGCGGTAATACGGTTATCCACAGAAT
CAGGGGATAACGCAGGAAAGAACATGTGAGCAAAAGGCCAGCAAAAGGCCAGGAAC
CGTAAAAAGGCCGCGTTGCTGGCGTTTTTCCATAGGCTCCGCCCCCCTGACGAGCATCA
CAAAAATCGACGCTCAAGTCAGAGGTGGCGAAACCCGACAGGACTATAAAGATACCA
GGCGTTTCCCCCTGGAAGCTCCCTCGTGCGCTCTCCTGTTCCGACCCTGCCGCTTACCG
GATACCTGTCCGCCTTTCTCCCTTCGGGAAGCGTGGCGCTTTCTCATAGCTCACGCTGT
AGGTATCTCAGTTCGGTGTAGGTCGTTCGCTCCAAGCTGGGCTGTGTGCACGAACCCCC
CGTTCAGCCCGACCGCTGCGCCTTATCCGGTAACTATCGTCTTGAGTCCAACCCGGTAA
GACACGACTTATCGCCACTGGCAGCAGCCACTGGTAACAGGATTAGCAGAGCGAGGTA
TGTAGGCGGTGCTACAGAGTTCTTGAAGTGGTGGCCTAACTACGGCTACACTAGAAGG
ACAGTATTTGGTATCTGCGCTCTGCTGAAGCCAGTTACCTTCGGAAAAAGAGTTGGTAG
CTCTTGATCCGGCAAACAAACCACCGCTGGTAGCGGTGGTTTTTTTGTTTGCAAGCAGC
AGATTACGCGCAGAAAAAAAGGATCTCAAGAAGATCCTTTGATCTTTTCTACGGGGTC
TGACGCTCAGTGGAACGAAAACTCACGTTAAGGGATTTTGGTCATGAGATTATCAAAA
AGGATCTTCACCTAGATCCTTTTAAATTAAAAATGAAGTTTTAAATCAATCTAAAGTAT
ATATGAGTAAACTTGGTCTGACAGTTACCAATGCTTAATCAGTGAGGCACCTATCTCAG
CGATCTGTCTATTTCGTTCATCCATAGTTGCCTGACTCCCCGTCGTGTAGATAACTACG
ATACGGGAGGGCTTACCATCTGGCCCCAGTGCTGCAATGATACCGCGAGACCCACGCT
CACCGGCTCCAGATTTATCAGCAATAAACCAGCCAGCCGGAAGGGCCGAGCGCAGAA
GTGGTCCTGCAACTTTATCCGCCTCCATCCAGTCTATTAATTGTTGCCGGGAAGCTAGA
GTAAGTAGTTCGCCAGTTAATAGTTTGCGCAACGTTGTTGCCATTGCTACAGGCATCGT
GGTGTCACGCTCGTCGTTTGGTATGGCTTCATTCAGCTCCGGTTCCCAACGATCAAGGC
GAGTTACATGATCCCCCATGTTGTGCAAAAAAGCGGTTAGCTCCTTCGGTCCTCCGATC
GTTGTCAGAAGTAAGTTGGCCGCAGTGTTATCACTCATGGTTATGGCAGCACTGCATAA
TTCTCTTACTGTCATGCCATCCGTAAGATGCTTTTCTGTGACTGGTGAGTACTCAACCA
AGTCATTCTGAGAATAGTGTATGCGGCGACCGAGTTGCTCTTGCCCGGCGTCAATACG
GGATAATACCGCGCCACATAGCAGAACTTTAAAAGTGCTCATCATTGGAAAACGTTCT
TCGGGGCGAAAACTCTCAAGGATCTTACCGCTGTTGAGATCCAGTTCGATGTAACCCA
CTCGTGCACCCAACTGATCTTCAGCATCTTTTACTTTCACCAGCGTTTCTGGGTGAGCA
AAAACAGGAAGGCAAAATGCCGCAAAAAAGGGAATAAGGGCGACACGGAAATGTTG
AATACTCATACTCTTCCTTTTTCAATATTATTGAAGCATTTATCAGGGTTATTGTCTCAT
GAGCGGATACATATTTGAATGTATTTAGAAAAATAAACAAATAGGGGTTCCGCGCACA
TTTCCCCGAAAAGTGCCACCTGACGTCTAAGAAACCATTATTATCATGACATTAACCTA
TAAAAATAGGCGTATCACGAGGCCCTTTCGTC

The following is a useful reference to the present invention, which is an article published by NATURE COMMUNICAITONS and discloses more detail examples and implements, thought published after the prior date of the present invention, the content of the article is can be taken as part of the BRIEF DESCRIPTION OF THE DRAWINGS of the present invention when considering the implement of the present invention:

Puping Liang, Xiaowei Xie, Shengyao Zhi, Hongwei Sun, Xiya Zhang, Yu Chen, Yuxi Chen, Yuanyan Xiong, Wenbin Ma, Dan Liu, Junjiu Huang & Zhou Songyang. Genome-wide profiling of adenine base editor specificity by EndoV-seq. NATURE COMMUNICAITONS (2019) 10:67|https://doi.org/10.1038/s41467-018-07988-z.

All references cited herein are incorporated by reference to the same extent as if each individual publication, database entry (e.g., Genbank sequences or GeneID entries), patent application, or patent, was specifically and individually indicated to be incorporated by reference. This statement of incorporation by reference is intended by Applicants to relate to each and every individual publication, database entry (e.g., Genbank sequences or GeneID entries), patent application, or patent identified even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents.

Finally, it should be noted that, the above embodiments are merely used for the convenience of describing the present disclosure and are not limited thereto. Although the present disclosure has been described in detail with reference to the foregoing embodiments, those skilled in the art should understand that the technical solutions described in the foregoing embodiments may be modified or equivalently substituted for some or all of the technical features. These modifications and substitutions do not depart from the scope of the technical solutions of the embodiments of the present invention.

What is claimed is:

1. A method for detecting the genome-wide off-target effects of adenine base editor, wherein, comprising the following steps:
(1) TadA:TadA*:Cas9 fusion protein, one or more kinds of gRNA targeting to DNA sequence to be detected, and genomic DNA comprising the DNA sequence to be detected are blended and then subjected to reaction; wherein,
in the reaction system, the DNA strand to be detected complementary to the gRNA is nicked by the TadA:TadA*:Cas9 fusion protein and gRNA complex, and the Adenine on the non-complementary strand is converted to Inosine;
(2) adding Endonuclease V into the system after reaction in the step (1), and cutting the DNA containing Inosine to cause double-strand DNA break;
(3) the off-target effects of the adenine base editor are detected by using whole-genome sequencing and bioinformatics analysis.

2. The method of claim 1, wherein the TadA:TadA*:Cas9 fusion protein comprises an effector protein domain of CRISPR/Cas9 system and an adenosine deaminase domain.

3. The method of claim 1, wherein the TadA:TadA*:Cas9 fusion protein comprises an effector protein domain of CRISPR/Cas9 system, a polypeptide linker and an adenosine deaminase domain.

4. The method of claim 1, wherein the TadA:TadA*:Cas9 fusion protein comprises an effector protein domain of CRISPR/Cas9 system, the Cas9 effector protein in the effector protein domain of the CRISPR/Cas system comprises, but is not limited to, one or more CAS proteins with no cleavage activity or only single strand cleavage activity, such as Streptococcus pyogenes Cas9, Staphylococcus aureus Cas9 Lachnospiraceae Cpf1 Acidaminococcus Cpf1, Streptococcus thermophilus Cas9, and Neisseria meningitidis Cas9 and Francisella Cpf1.

5. The method of claim 1, wherein the TadA:TadA*:Cas9 fusion protein comprises a adenosine deaminase TadA protein, the amino acid sequence of the adenosine deaminase TadA protein comprises SEQ ID NO.1.

6. The method of claim 1, wherein the amino acid sequence of the TadA:TadA*:Cas9 fusion protein comprises SEQ ID NO.2 or a sequence consistent with at least 80%, 85%, 90%, 92%, 95%, 96%, 97%, 98%, 99% or 99.5% of the amino acid sequence shown in SEQ ID NO.2.

7. The method of claim 1, wherein the TadA:TadA*:Cas9 fusion protein is expressed in bacteria containing expression vector and then purified.

8. The method of claim 1, wherein the reaction system is a solution reaction system, and the solution reaction system further comprises buffer solution components required for converting Adenine on the non-complementary strand into Inosine by the TadA:TadA*:Cas9 fusion protein.

9. The method of claim 1, wherein the step (3) comprises:
performing whole genome sequencing on the production subjected to enzyme digestion in the step (2) to obtain a whole-genome sequencing result;
performing bioinformatics analysis on the whole-genome sequencing result to obtain off-target data of adenine base editor.

10. The method of claim 9, wherein the step (3) further comprises: predicting the off-target effects of adenine base editor in cells or in body according to the off-target data.

11. The method of claim 10, wherein the cells include human cells, animal cells or plant cells.

12. The method of claim 10, wherein the body includes humans, animals or plants.

13. A kit for detecting the genome-wide off-target effects of adenine base editor, wherein comprises the gRNA targeting DNA to be detected, TadA:TadA*:Cas9 fusion protein or the Endonuclease V nuclease of claim 1.

14. A method of claim 1, wherein, the efficiency of detecting the off-target effects of adenine base editor can be at least low to 0.13%.

15. A method of claim 1, wherein, applying the method of claim 1 in gene editing.
